(19) United States
(12) Patent Application Publication
(10) Pub. No.: US 2025/0085282 A1
(43) Pub. Date: Mar. 13, 2025

(54) METHOD FOR DETECTING ANTI-SARS-COV-2 SPIKE (S) IMMUNOGLOBULINS

(71) Applicant: NOVAVAX, INC., Gaithersburg, MD (US)

(72) Inventors: Joyce S. PLESTED, Gaithersburg, MD (US); Raj KALKERI, Gaithersburg, MD (US)

(21) Appl. No.: 18/882,452

(22) Filed: Sep. 11, 2024

Related U.S. Application Data
(60) Provisional application No. 63/581,754, filed on Sep. 11, 2023.

(51) Int. Cl. G01N 33/569

(52) U.S. Cl. CPC G01N 33/56983; G01N 2333/165; G01N 2469/20

(57) ABSTRACT

Disclosed are methods for detecting if a biological sample (e.g., serum, blood, plasma) contains antibodies against SARS-CoV-2 S glycoproteins comprising: (i) providing a surface coated with a SARS-CoV-2 S glycoprotein; (ii) exposing the surface to the biological sample; (iii) exposing the surface to a secondary antibody; and (iv) detecting the secondary antibody that is bound to the surface; wherein the biological sample contains antibodies that bind to the SARS-CoV-2 S glycoprotein if secondary antibody is detected.

### CROSS REFERENCE TO RELATED APPLICATIONS

This application claims priority to U.S. Provisional Application No. 63/581,754 filed on Sep. 11, 2023. The aforementioned application is incorporated by reference herein in its entirety.

### REFERENCE TO AN ELECTRONIC SEQUENCE LISTING

This application contains a Sequence Listing which has been submitted electronically in XML format and is hereby incorporated by reference in its entirety. Said XML copy, created on Sep. 11, 2024 is named 1450_105US1_Sequence_Listing_09_11_2024 and is 814,987 bytes in size.

### FIELD

The present disclosure is generally related to methods for identifying if a biological sample (e.g., serum, blood, plasma) contains antibodies against SARS-CoV-2 S glycoproteins.

### BACKGROUND OF THE INVENTION

The coronavirus disease 2019 (COVID-19) pandemic is caused by severe acute respiratory syndrome coronavirus 2 (SARS-CoV-2). The emergence of variants (such as Alpha, Beta, Gamma, Delta, and multiple Omicron subvariants) has led to ongoing transmission of the virus. Some SARS-CoV-2 variants (such as Omicron) have immune evasion properties, thus reducing the effectiveness of COVID-19 vaccines. There is a need for additional correlates of protection (CoPs) for COVID vaccine efficacy to help track immune evasion and understand the needs of the vaccine development landscape (e.g., development of new vaccines based on variant S protein sequences). Validated CoPs help to extrapolate vaccine efficacy/immunogenicity results to populations or formulations/schedules not represented in clinical trials. Of particular interest are CoPs for durability and level of vaccine-driven protection against ancestral and variant strains.

Biomarkers of immunogenicity, which may eventually be proven to be correlates of protection, are critical for assessment of vaccines. In particular, the amount of anti-SARS-CoV-2 S (spike) antibodies in a biological sample determines the effectiveness of the humoral response induced by the vaccine. Current assays measure total anti-SARS-CoV-2 S antibodies. Not all of these antibodies are desirable. Improved assays to evaluate anti-SARS-CoV-2 S antibodies induced by vaccines is necessary.

There are four unique subclasses of human IgG, numbered in order of relative abundance, include IgG1, IgG2, IgG3, and IgG4. IgG1 and IgG3 are the major contributors to rapid IgG responses to protein and membrane antigens, and are active in viral neutralization, although some differences in IgG subclass responses have been identified in the context of SARS-CoV-2 infection and vaccination. Indeed, IgG3 response to SARS-CoV-2 infection has been highlighted by an analysis of convalescent plasma, showing that while IgG3 accounted for 12% of total anti-Spike (anti-S) protein IgG, it contributed to approximately 80% of total live SARS-CoV-2 neutralizing activity. In addition, IgG1, and especially IgG3, bind FcγRIIa, FcγRIIIa, and C1q, thereby supporting antibody-dependent cellular phagocytosis (ADCP), antibody-dependent cytotoxicity (ADCC), and antibody-dependent complement deposition (ADCD).

Repeated mRNA SARS-CoV-2 vaccination has been associated with significant increases in the proportion of immunoglobulin G4 (IgG4) in Spike-specific responses and reductions in Fc-mediated ADCP and ADCD that may limit control of viral infection. After repeated mRNA SARS-CoV-2 vaccination, IgG3 was observed to reach peak levels after a second dose, and steadily declined thereafter to significantly lower levels after the third and fourth dose vaccinations, while IgG4 increased. ₅IgG4 is generally in low abundance (0-5% of total IgG) but may slowly increase over time due to repeated or excessive exposure to some antigens. Although repeated antigen exposure seems necessary, it is insufficient to induce IgG4, as prolonged activation of IL-10-expressing CD4⁺ T-cells or expression of other anti-inflammatory cytokines is also required. Increased concentrations of IgG4 have been associated with immunosuppression and poor clinical outcomes of COVID-19, and while generally regarded as anti-inflammatory, may contribute to some autoimmune disorders and inflammatory IgG4-related diseases.

### SUMMARY OF THE INVENTION

Provided herein are methods for determining if a biological sample contains antibodies that bind to the SARS-CoV-2 Spike (S) glycoprotein, comprising: (i) providing a surface coated with a SARS-CoV-2 S glycoprotein; (ii) exposing the surface to the biological sample; (iii) exposing the surface to a secondary antibody; and (iv) detecting the secondary antibody that is bound to the surface; wherein the biological sample contains antibodies that bind to the SARS-CoV-2 S glycoprotein if secondary antibody is detected. In embodiments, the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 2, 4, 38, 41, 44, 48, 51, 54, 58, 61, 63, 65, 67, 73, 75, 78, 79, 82, 83, 85, 106, 108, 89, and 110, 112-115, 132, 133, 114, 138, 141, 144, 147, 151, 153, 156, 158, 174, 175, 176, 181-184, 186, 188, 190, 195, 217-228, 233-236, 243, 255-264, 273-280, 283, 284, 287, 288, 291, 292, and 294, 296, 298, 300, 302, 304, 306, 308, 310, 312, 314, 316, 318, 320, 322, 324, 236, 328, 329, 330, 331, 332 and 333. In embodiments, the SARS-CoV-2 S glycoprotein has an inactive furin cleavage site. In embodiments, the inactive furin cleavage site comprises the amino acid sequence of QQAQ (SEQ ID NO: 7). In embodiments, amino acids 973 and 974 of the SARS-CoV-2 S glycoprotein are proline, as compared to a wild-type SARS-CoV-2 S glycoprotein having the amino acid sequence of SEQ ID NO: 2. In embodiments, the antibodies that bind to the SARS-CoV-2 S glycoprotein are IgG. In embodiments, the antibodies that bind to the SARS-CoV-2 S glycoprotein are IgG1. In embodiments, the antibodies that bind to the SARS-CoV-2 S glycoprotein are IgG2. In embodiments, the antibodies that bind to the SARS-CoV-2 S glycoprotein are IgG3. In embodiments, the antibodies that bind to the SARS-CoV-2 S glycoprotein are IgG4. In embodiments, the secondary antibody is selected from the group consisting of an anti-human IgG antibody, an anti-human IgG1 antibody, an anti-human IgG2 antibody, an anti-human IgG3 antibody, and an anti-human IgG4 antibody. In embodiments, the SARS-CoV-2 S glycoprotein is from a SARS-CoV-2 virus or a variant of SARS-CoV-2. In embodiments, the variant of SARS-CoV-2 is a B.1.1.7 SARS-CoV-2 strain; a B.1.351 SARS-CoV-2 strain; a P.1 SARS-CoV-2 strain; a Cal.20C SARS-CoV-2 strain; a B.1.617.2 SARS-CoV-2 strain; a B.1.525 SARS-CoV-2 strain; a B.1.526 SARS-CoV-2 strain; a B.1.617.1 SARS-CoV-2 strain; a C.37 SARS-CoV-2 strain; a B.1.621 SARS-CoV-2 strain; or a B.1.1.529 SARS-CoV-2 strain. In embodiments, the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a SARS-CoV-2 S glycoprotein from a SARS-CoV-2 S omicron variant selected from the group consisting of: BA.1, BA.2.12.1, BA.2, BA.3, BA.4, BA.5, XBB.1.5, XBB.2.3, XBB.1.16, EG.5.1, JN.1, BQ.1.1, BF.7. In embodiments, the secondary antibody is attached to a tag. In embodiments, the tag is horseradish peroxidase. In embodiments, the biological sample is serum, plasma, blood, saliva, a nasopharyngeal swab, or mucus. In embodiments, the biological sample is from a patient that has previously had COVID-19. In embodiments, the biological sample is from a patient that has been administered an immunogenic composition against a SARS-CoV-2 virus or a variant thereof. In embodiments, the SARS-CoV-2 S glycoprotein includes the transmembrane domain. In embodiments, evaluating binding IgG responses to SARS-CoV-2 S glycoprotein in serum results in rapid development of effective vaccines against emerging SARS-CoV-2 variants.

### BRIEF DESCRIPTION OF THE DRAWINGS

The invention may be more completely understood in consideration of the following detailed description of various embodiments of the invention in connection with the accompanying drawings, in which:

FIG. 1 presents schematics of the procedure for performing an anti-rS IgG detection assay;

FIGS. 2A-2C are schematics of assays that show how to detect anti-spike IgG4 (FIG. 2A), anti-spike IgG1 (FIG. 2B) and total anti-spike IgG (FIG. 2C);

FIG. 3 shows the % of IgG1 out total IgG in the samples at day 1 (before boost) and at day 29 (28 days after boost);

FIG. 4 shows the % of IgG4 out total IgG in the samples at day 1 (before boost) and at day 29 (28 days after boost);

FIG. 5 shows the % of IgG4 out total IgG in the samples at day 29 (28 days after boost);

FIG. 6 shows the % of IgG1 and IgG4 in the samples at day 1 (before boost);

FIG. 7 shows the % of IgG1 and IgG4 in the samples at day 29 (28 days after boost);

FIG. 8 shows the total anti-S glycoprotein IgG in the samples;

FIG. 9 shows the total anti-S glycoprotein IgG1 in the samples;

FIG. 10 shows the total anti-S glycoprotein IgG4 in the samples.

FIG. 11 shows inter-assay, intra-assay, and total precision were <20% GCV for all 27 serum samples representing all concentration ranges (low, medium, and high concentrations);

FIG. 12 shows IgG assay ancestral strain assay specificity;

FIG. 13 shows IgG assay ancestral strain hemoglobin and lipemic matrix effects;

FIGS. 14A-14B show IgG assay ancestral strain linearity;

FIG. 15 shows IgG assay ancestral strain temperature and freeze/thaw stability;

FIGS. 16A-16F show IgG assay validation parameters for the Beta (FIG. 16B), Delta (FIG. 16C), and Omicron BA.1 (FIG. 16D), BA.5 (FIG. 16E), XBB.1.5 (FIG. 16F) variants were similar to those for the ancestral strain (FIG. 16A);

FIGS. 17A-17F show correlation of IgG assay results with other immunogenicity assays in SARS-CoV-2 seronegative serum samples;

FIGS. 18A-18F show correlation of IgG assay results with other immunogenicity assays in SARS-CoV-2 seropositive serum samples;

FIG. 19A shows IgG subclass serum concentrations after repeated SARS-CoV-2 vaccination;

FIG. 19B shows Surrogate Fc effector function responses after repeated SARS-CoV-2 vaccination.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF THE INVENTION

### Definitions

As used herein, and in the appended claims, the singular forms “a”, “an”, and “the” include plural referents unless the context clearly dictates otherwise. Thus, for example, reference to “a protein” can refer to one protein or to mixtures of such protein, and reference to “the method” includes reference to equivalent steps and/or methods known to those skilled in the art, and so forth.

As used herein, the term “adjuvant” refers to a compound that, when used in combination with an immunogen, augments or otherwise alters or modifies the immune response induced against the immunogen. Modification of the immune response may include intensification or broadening the specificity of either or both antibody and cellular immune responses.

As used herein, the term “about” or “approximately” when preceding a numerical value indicates the value plus or minus a range of 10%. For example, “about 100” encompasses 90 and 110. As used herein, an “immunogenic composition” is a composition that comprises an antigen where administration of the composition to a subject results in the development in the subject of a humoral and/or a cellular immune response to the antigen.

As used herein, a “subunit” composition, for example a vaccine, that includes one or more selected antigens but not all antigens from a pathogen. Such a composition is substantially free of intact virus or the lysate of such cells or particles and is typically prepared from at least partially purified, often substantially purified immunogenic polypeptides from the pathogen. The antigens in the subunit composition disclosed herein are typically prepared recombinantly, often using a baculovirus system.

As used herein, “substantially” refers to isolation of a substance (e.g. a compound, polynucleotide, or polypeptide) such that the substance forms the majority percent of the sample in which it is contained. For example, in a sample, a substantially purified component comprises 85%, preferably 85%-90%, more preferably at least 95%-99.5%, and most preferably at least 99% of the sample. If a component is substantially replaced the amount remaining in a sample is less than or equal to about 0.5% to about 10%, preferably less than about 0.5% to about 1.0%.

The terms “treat,” “treatment,” and “treating,” as used herein, refer to an approach for obtaining beneficial or desired results, for example, clinical results. For the purposes of this disclosure, beneficial or desired results may include inhibiting or suppressing the initiation or progression of an infection or a disease; ameliorating, or reducing the development of, symptoms of an infection or disease; or a combination thereof.

“Prevention,” as used herein, is used interchangeably with “prophylaxis” and can mean complete prevention of an infection or disease, or prevention of the development of symptoms of that infection or disease; a delay in the onset of an infection or disease or its symptoms; or a decrease in the severity of a subsequently developed infection or disease or its symptoms.

As used herein an “effective dose” or “effective amount” refers to an amount of an immunogen sufficient to induce an immune response that reduces at least one symptom of pathogen infection. An effective dose or effective amount may be determined e.g., by measuring amounts of neutralizing secretory and/or serum antibodies, e.g., by plaque neutralization, complement fixation, enzyme-linked immunosorbent (ELISA), or microneutralization assay.

As used herein, the term “vaccine” refers to an immunogenic composition, such as an immunogen derived from a pathogen, which is used to induce an immune response against the pathogen. The immune response may include formation of antibodies and/or a cell-mediated response. Depending on context, the term “vaccine” may also refer to a suspension or solution of an immunogen that is administered to a subject to produce an immune response. Preferably, vaccines induces an immune response that is effective at preventing infection from SARS-CoV-2 or a variant thereof.

As used herein, the term “subject” includes humans and other animals. Typically, the subject is a human. For example, the subject may be an adult, a teenager, a child (2 years to 14 years of age), an infant (birth to 2 year), or a neonate (up to 2 months). In particular aspects, the subject is up to 4 months old, or up to 6 months old. In aspects, the adults are seniors about 65 years or older, or about 60 years or older. In aspects, the subject is a pregnant woman or a woman intending to become pregnant. In other aspects, subject is not a human; for example a non-human primate; for example, a baboon, a chimpanzee, a gorilla, or a macaque. In certain aspects, the subject may be a pet, such as a dog or cat.

As used herein, the term “pharmaceutically acceptable” means being approved by a regulatory agency of a U.S. Federal or a state government or listed in the U.S. Pharmacopeia, European Pharmacopeia or other generally recognized pharmacopeia for use in mammals, and more particularly in humans. These compositions can be useful as a vaccine and/or antigenic compositions for inducing a protective immune response in a vertebrate.

As used herein, the term “NVX-CoV2373” refers to a vaccine composition comprising the BV2373 Spike glycoprotein (SEQ ID NO: 87) and Fraction A and Fraction C iscom matrix (e.g., MATRIX-M™).

As used herein, the term “modification” as it refers to a CoV S polypeptide refers to mutation, deletion, or addition of one amino acid of the CoV S polypeptide. The location of a modification within a CoV S polypeptide can be determined based on aligning the sequence of the polypeptide to SEQ ID NO: 1 (CoV S polypeptide containing signal peptide) or SEQ ID NO: 2 (mature CoV S polypeptide lacking a signal peptide).

### Assays for Evaluating the Immunogenicity of Vaccine Compositions Against SARS-CoV-2

The disclosure provides assays for evaluating the immunogenicity of immunogenic compositions and vaccine compositions against SARS-CoV-2. In embodiments, the assay evaluates the amount of anti-rS proteins IgG1, IgG2, IgG3, and IgG4, and total anti-rS IgG in a biological sample. (see Example 1). Surrogate ADCP (FcγRIIa binding), surrogate ADCC (FcγRIIIa binding), and ADCD (C1q binding) in a biological sample are also evaluated.

In embodiments, the immunogenic compositions and vaccine compositions contain non-naturally occurring coronavirus (CoV) Spike (5) polypeptides or nanoparticles containing CoV S polypeptides.

In embodiments, the CoV S polypeptide comprises a sequence that is at least 90%, at least 91%, at least 92%, at least 93%, at least 94%, at least 95%, at least 96%, at least 97%, at least 98%, at least 99%, or 100% identical to SEQ ID NO: 87. The sequence of SEQ ID NO: 87 is in the table below,

SEQ ID NO: 87
QCVNLTTRTQLPPAYTNSFTRGVYYPDKVFRSSVLHSTQDLFLPFFSNVT
WFHAIHVSGTNGTKRFDNPVLPFNDGVYFASTEKSNIIRGWIFGTTLDSK
TQSLLIVNNATNVVIKVCEFQFCNDPFLGVYYHKNNKSWMESEFRVYSSA
NNCTFEYVSQPFLMDLEGKQGNFKNLREFVFKNIDGYFKIYSKHTPINLV
RDLPQGFSALEPLVDLPIGINITRFQTLLALHRSYLTPGDSSSGWTAGAA
AYYVGYLQPRTFLLKYNENGTITDAVDCALDPLSETKCTLKSFTVEKGIY
QTSNFRVQPTESIVRFPNITNLCPFGEVFNATRFASVYAWNRKRISNCVA
DYSVLYNSASFSTFKCYGVSPTKLNDLCFTNVYADSFVIRGDEVRQIAPG
QTGKIADYNYKLPDDFTGCVIAWNSNNLDSKVGGNYNYLYRLFRKSNLKP
FERDISTEIYQAGSTPCNGVEGFNCYFPLQSYGFQPTNGVGYQPYRVVVL
SFELLHAPATVCGPKKSTNLVKNKCVNFNFNGLTGTGVLTESNKKFLPFQ
QFGRDIADTTDAVRDPQTLEILDITPCSFGGVSVITPGTNTSNQVAVLYQ
DVNCTEVPVAIHADQLTPTWRVYSTGSNVFQTRAGCLIGAEHVNNSYECD
IPIGAGICASYQTQTNSPQQAQSVASQSIIAYTMSLGAENSVAYSNNSIA
IPTNFTISVTTEILPVSMTKTSVDCTMYICGDSTECSNLLLQYGSFCTQL
NRALTGIAVEQDKNTQEVFAQVKQIYKTPPIKDFGGFNFSQILPDPSKPS
KRSFIEDLLENKVTLADAGFIKQYGDCLGDIAARDLICAQKFNGLTVLPP
LLTDEMIAQYTSALLAGTITSGWTFGAGAALQIPFAMQMAYRENGIGVTQ
NVLYENQKLIANQFNSAIGKIQDSLSSTASALGKLQDVVNQNAQALNTLV
KQLSSNFGAISSVLNDILSRLDPPEAEVQIDRLITGRLQSLQTYVTQQLI
RAAEIRASANLAATKMSECVLGQSKRVDFCGKGYHLMSFPQSAPHGVVFL
HVTYVPAQEKNFTTAPAICHDGKAHFPREGVFVSNGTHWFVTQRNFYEPQ
IITTDNTFVSGNCDVVIGIVNNTVYDPLQPELDSFKEELDKYFKNHTSPD
VDLGDISGINASVVNIQKEIDRLNEVAKNLNESLIDLQELGKYEQYIKWP
WYIWLGFIAGLIAIVMVTIMLCCMTSCCSCLKGCCSCGSCCKFDEDDSEP
VLKGVKLHYT

In embodiments, the methods further comprise washing the solid surface. In embodiments, the methods comprise contacting the plate with a blocking buffer. A blocking buffer is a solution that removes the possibility of non-specific binding to the plate.

In embodiments, the methods may be utilized to detect antibody isotypes selected from the group consisting of IgA, IgG, IgM, IgD, and IgE. In embodiments, the methods may be utilized to detect antibody isotypes selected from the group consisting of IgG1, IgG2, IgG3, and IgG4.

### SARS-COV-2 S Glycoproteins for Use in the Methods

In embodiments, suitable SARS-COV-2 S glycoproteins for use in the methods described herein include the SARS-COV-2 S glycoproteins associated with Protein Data Bank (PDB) IDs of any one of: 6LVN, 6LZG, 6MOJ, 6M17, 6M1V, 6VSB, 6VW1, 6VXX, 6VYB, 6W41, 6WPS, 6WPT, 6X29, 6X2A, 6 X2B, 6X2C, 6X45, 6X6P, 6X79, 6XC2, 6XC3, 6XC4, 6XC7, 6XCM, 6XCN, 6XDG, 6XE1, 6XEY, 6XF 5, 6XF6, 6XKL, 6XKP, 6XKQ, 6XLU, 6XM0, 6XM3, 6XM4, 6XM5, 6XR8, 6XRA, 6XS6, 6YBB, 6YL A, 6YM0, 6YOR, 6YZ5, 6YZ7, 6Z2M, 6Z43, 6Z97, 6ZB4, 6ZB5, 6ZBP, 6ZCZ, 6ZDG, 6ZDH, 6ZER, 6Z FO, 6ZGE, 6ZGG, 6ZGH, 6ZGI, 6ZH9, 6ZHD, 6ZLR, 6ZOW, 6ZOX, 6ZOY, 6ZOZ, 6ZP0, 6ZP1, 6ZP2, 6ZP5, 6ZP7, 6ZWV, 6ZXN, 7A25, 7A29, 7A4N, 7A5R, 7A5S, 7A91, 7A92, 7A93, 7A94, 7A95, 7A96, 7A97, 7A98, 7AD1, 7AKD, 7BOB, 7B14, 7B17, 7B18, 7B27, 7B30, 7B62, 7BEH, 7BEI, 7BEJ, 7BEK, 7B EL, 7BEM, 7BEN, 7BEO, 7BEP, 7BH9, 7BNM, 7BNN, 7BNO, 7BNV, 7BWJ, 7BYR, 7BZ5, 7C01, 7C2 L, 7C53, 7C8D, 7C8J, 7C8V, 7C8W, 7CAB, 7CAC, 7CAH, 7CAI, 7CAK, 7CAN, 7CDI, 7CDJ, 7CH4, 7 CH5, 7CHB, 7CHC, 7CHE, 7CHF, 7CHH, 7CHO, 7CHP, 7CHS, 7CJF, 7CM4, 7CN9, 7COT, 7CT5, 7C WL, 7CWM, 7CWN, 7CWO, 7CWS, 7CWT, 7CWU, 7CYH, 7CYP, 7CYV, 7CZP, 7CZQ, 7CZR, 7CZS, 7CZT, 7CZU, 7CZV, 7CZW, 7CZX, 7CZY, 7CZZ, 7D00, 7D03, 7DOB, 7DOC, 7DOD, 7D2Z, 7D30, 7D 4G, 7D61, 7DCC, 7DCX, 7DD2, 7DD8, 7DDD, 7DDN, 7DE0, 7DET, 7DEU, 7DF3, 7DF4, 7DHX, 7DJ Z, 7DK0, 7DK2, 7DK3, 7DK4, 7DK5, 7DK6, 7DK7, 7DMU, 7DPM, 7DQA, 7DTE, 7DWX, 7DWY, 7D WZ, 7DX0, 7DX1, 7DX2, 7DX3, 7DX4, 7DX5, 7DX6, 7DX7, 7DX8, 7DX9, 7DZW, 7DZX, 7DZY, 7E2 3, 7E39, 7E3B, 7E3C, 7E3J, 7E3K, 7E3L, 7E30, 7E50, 7E5R, 7E5S, 7E5Y, 7E7B, 7E7D, 7E7X, 7E7Y, 7E86, 7E88, 7E8C, 7E8F, 7E8M, 7E9N, 7E90, 7E9P, 7E9Q, 7E9T, 7EAM, 7EAN, 7EAZ, 7EB0, 7EB3, 7 EB4, 7EB5, 7EDF, 7EDG, 7EDH, 7EDI, 7EDJ, 7EFP, 7EFR, 7EH5, 7EJ4, 7EJ5, 7EJL, 7EJY, 7EJZ, 7EK 0, 7EK6, 7EKC, 7EKE, 7EKF, 7EKG, 7EKH, 7ENF, 7ENG, 7EPX, 7EY0, 7EY4, 7EY5, 7EYA, 7EZV, 7F0X, 7F12, 7F15, 7F3Q, 7F46, 7F5H, 7F5R, 7F62, 7F63, 7F6Y, 7F6Z, 7F7E, 7F7H, 7FAE, 7FAF, 7FAT, 7FAU, 7FB0, 7FB1, 7FB3, 7FB4, 7FBJ, 7FBK, 7FC5, 7FCD, 7FCE, 7FCP, 7FCQ, 7FDG, 7FDH, 7FDI, 7FDK, 7FEM, 7FET, 7FG2, 7FG3, 7FG7, 7FH0, 7FJC, 7FJN, 7FJO, 7FJS, 7JJC, 7JJI, 7JJJ, 7JMO, 7JMP, 7JMW, 7JV2, 7JV4, 7JV6, 7JVA, 7JVB, 7JVC, 7JW0, 7JWB, 7JWY, 7JX3, 7JZL, 7JZM, 7JZN, 7JZU, 7K43, 7K45, 7K4N, 7K8M, 7K8S, 7K8T, 7K8U, 7K8V, 7K8W, 7K8X, 7K8Y, 7K8Z, 7K90, 7K9H, 7K9I, 7K9J, 7K9K, 7K9Z, 7KDG, 7KDH, 7KDI, 7KDJ, 7KDK, 7KDL, 7KE4, 7KE6, 7KE7, 7KE8, 7KE9, 7K EA, 7KEB, 7KEC, 7KFV, 7KFW, 7KFX, 7KFY, 7KGJ, 7KGK, 7KJ2, 7KJ3, 7KJ4, 7KJ5, 7KKK, 7KKL, 7KL9, 7KLG, 7KLH, 7KLW, 7KM5, 7KMB, 7KMG, 7KMH, 7KMI, 7KMK, 7KML, 7KMS, 7KMZ, 7 KN3, 7KN4, 7KN5, 7KN6, 7KN7, 7KNB, 7KNE, 7KNH, 7KNI, 7KQE, 7KRQ, 7KRR, 7KRS, 7KS9, 7 KSG, 7KXJ, 7KXK, 7KZB, 7L02, 7L06, 7L09, 7LON, 7L2C, 7L2D, 7L2E, 7L2F, 7L3N, 7L4Z, 7L56, 7L 57, 7L58, 7L5B, 7L7D, 7L7E, 7L7F, 7L7K, 7LAA, 7LAB, 7LC8, 7LCN, 7LD1, 7LDJ, 7LJR, 7LM8, 7L O4, 7LOP, 7LQ7, 7LQV, 7LQW, 7LRS, 7LRT, 7LS9, 7LSS, 7LWI, 7LWJ, 7LWK, 7LWL, 7LWM, 7L WN, 7LWO, 7LWP, 7LWQ, 7LWS, 7LWT, 7LWU, 7LWV, 7LWW, 7LX5, 7LXW, 7LXX, 7LXY, 7LX Z, 7LY0, 7LY2, 7LY3, 7LYK, 7LYL, 7LYM, 7LYN, 7LY0, 7LYP, 7LYQ, 7MOJ, 7M31, 7M42, 7M53, 7M6D, 7M6E, 7M6F, 7M6G, 7M6H, 7M61, 7M71, 7M7B, 7M7W, 7M8J, 7M8K, 7M8S, 7M8T, 7M8U, 7 MDW, 7ME7, 7MEJ, 7MF1, 7MFU, 7MJG, 7MJH, 7MJI, 7MJJ, 7MJK, 7MJL, 7MJM, 7MJN, 7MKL, 7 MKM, 7MLZ, 7MM0, 7MMO, 7MSQ, 7MTC, 7MTD, 7MTE, 7MW2, 7MW3, 7MW4, 7MW5, 7MW6, 7MY2, 7MY3, 7MY8, 7MZF, 7MZG, 7MZH, 7MZI, 7MZJ, 7MZK, 7MZL, 7MZM, 7MZN, 7NOG, 7NO H, 7NIA, 7NIB, 7NIE, 7NIF, 7NIQ, 7NIT, 7NIU, 7NIV, 7NIW, 7NIX, 7N1Y, 7N31, 7N41, 7N4J, 7N 4L, 7N4M, 7N5H, 7N62, 7N64, 7N6D, 7N6E, 7N8H, 7N81, 7N9A, 7N9B, 7N9C, 7N9E, 7N9T, 7NAB, 7 ND3, 7ND4, 7ND5, 7ND6, 7ND7, 7ND8, 7ND9, 7NDA, 7NDB, 7NDC, 7NDD, 7NEG, 7NEH, 7NKT, 7 NLL, 7NP1, 7NS6, 7NT9, 7NTA, 7NTC, 7NX6, 7NX7, 7NX8, 7NX9, 7NXA, 7NXB, 7NXC, 70AN, 70A0, 70AP, 70AQ, 70AU, 70AY, 7OD3, 7ODL, 7OLZ, 7OR9, 7ORA, 7ORB, 70WX, 7P19, 7P3D, 7P 40, 7P5G, 7P5Q, 7P5S, 7P77, 7P78, 7P79, 7P7A, 7P7B, 7PBE, 7PHG, 7PQY, 7PQZ, 7PR0, 7PRY, 7PRZ, 7PS0, 7PS1, 7PS2, 7PS4, 7PS5, 7PS6, 7PS7, 7Q0A, 7Q0G, 7Q0H, 7Q01, 7QIZ, 7Q3Q, 7Q3R, 7Q6E, 7Q 9F, 7Q9G, 7Q91, 7Q9J, 7Q9K, 7Q9M, 7Q9P, 7QEZ, 7QF0, 7QF1, 7QNW, 7QNX, 7QNY, 7Q07, 7Q09, 7QTI, 7QTJ, 7QTK, 7QUR, 7QUS, 7R0Z, 7R10, 7R11, 7R12, 7R13, 7R14, 7R15, 7R16, 7R17, 7R18, 7R 19, 7R1A, 7R1B, 7R40, 7R41, 7R4Q, 7R4R, 7R6W, 7R6X, 7R7N, 7R8L, 7R8M, 7R8N, 7R80, 7R95, 7R A8, 7RAL, 7RAQ, 7RBU, 7RBV, 7RBY, 7RKU, 7RKV, 7RNJ, 7RPV, 7RQ6, 7RR0, 7RTD, 7RTR, 7R U1, 7RU2, 7RU3, 7RU4, 7RU5, 7RU8, 7RW2, 7RXD, 7RZQ, 7RZR, 7RZS, 7RZT, 7RZU, 7RZV, 7S0B, 7S0C, 7SOD, 7SOE, 7S3N, 7S4S, 7S5P, 7S5Q, 7S5R, 7S61, 7S6J, 7S6K, 7S6L, 7S83, 7SA2, 7SBK, 7SB L, 7SBO, 7SBP, 7SBQ, 7SBR, 7SBS, 7SBT, 7SBU, 7SC1, 7SD5, 7SI2, 7SIS, 7SIX, 7SJ0, 7SJS, 7SKZ, 7 SL5, 7SN0, 7SN2, 7SN3, 7SO9, 7SOA, 7S0B, 7S0C, 7SOD, 7SOE, 7SOF, 7SPO, 7SPP, 7SWN, 7SWO, 7SWP, 7SWW, 7SWX, 7SXR, 7SXS, 7SXT, 7SXU, 7SXV, 7SXW, 7SXX, 7SXY, 7SXZ, 7SY0, 7SY1, 7SY2, 7SY3, 7SY4, 7SY5, 7SY6, 7SY7, 7SY8, 7T01, 7T3M, 7T67, 7T72, 7T7B, 7T9J, 7T9K, 7T9L, 7T AS, 7TAT, 7TB4, 7TB8, 7TBF, 7TCA, 7TCC, 7TCQ, 7TEI, 7TEW, 7TEX, 7TEY, 7TEZ, 7TF0, 7TF1, 7 TF2, 7TF3, 7TF4, 7TF5, 7TF8, 7TGE, 7TGW, 7TGX, 7TGY, 7THE, 7THK, 7THT, 7TIK, 7TL1, 7TL9, 7 TLA, 7TLB, 7TLC, 7TLD, 7TLT, 7TLY, 7TM0, 7TN0, 7TNW, 7TO4, 7TOU, 7TOV, 7TOW, 7TOX, 7T OY, 7TOZ, 7TP0, 7TP1, 7TP2, 7TP3, 7TP4, 7TP7, 7TP8, 7TP9, 7TPA, 7TPC, 7TPE, 7TPF, 7TPH, 7TPK, 7TPL, 7TPR, 7TYZ, 7TZ0, 7U09, 7U0A, 7UOD, 7UOE, 7UON, 7UOP, 7UOQ, 7U0X, 7UIR, 7U2D, 7U2E, 7U8E, 7U90, 7U9P, 7UAP, 7UAQ, 7UAR, 7UB0, 7UB5, 7UB6, 7UFK, 7UFL, 7UHC, 7UL0, 7UM2, 7UPL, 7UR1, 7URQ, 7URS, 7UZ4, 7UZ5, 7UZ6, 7UZ7, 7UZ8, 7UZ9, 7UZA, 7UZB, 7UZC, 7UZD, 7V 20, 7V22, 7V23, 7V24, 7V26, 7V27, 7V2A, 7V76, 7V77, 7V78, 7V79, 7V7A, 7V7D, 7V7E, 7V7F, 7V7 G, 7V7H, 7V71, 7V7J, 7V7N, 7V70, 7V7P, 7V7Q, 7V7R, 7V7S, 7V7T, 7V7U, 7V7V, 7V7Z, 7V80, 7V8 1, 7V82, 7V83, 7V84, 7V85, 7V86, 7V87, 7V88, 7V89, 7V8A, 7V8B, 7V8C, 7VHH, 7VHJ, 7VHK, 7VH L, 7VHM, 7VHN, 7VMU, 7VNB, 7VNC, 7VND, 7VNE, 7VOA, 7VQ0, 7VRV, 7VRW, 7VX1, 7VX4, 7 VX5, 7VX9, 7VXA, 7VXB, 7VXC, 7VXD, 7VXE, 7VXF, 7VX1, 7VXK, 7VXM, 7VYR, 7VZT, 7WIS, 7W6U, 7W8S, 7W92, 7W94, 7W99, 7W9B, 7W9C, 7W9E, 7W9F, 7WA1, 7WB5, 7WBL, 7WBP, 7WB Q, 7WBZ, 7WCD, 7WCH, 7WCK, 7WCP, 7WCR, 7WCU, 7WCZ, 7WD0, 7WD1, 7WD2, 7WD7, 7W D8, 7WD9, 7WDF, 7WE7, 7WE8, 7WE9, 7WEA, 7WEB, 7WEC, 7WED, 7WEE, 7WEF, 7WEV, 7WG 6, 7WG7, 7WG8, 7WG9, 7WGB, 7WGC, 7WGV, 7WGX, 7WGY, 7WGZ, 7WH8, 7WHB, 7WHD, 7W HH, 7WHI, 7WHJ, 7WHK, 7WHZ, 7WJY, 7WJZ, 7WK0, 7WK2, 7WK3, 7WK4, 7WK5, 7WK6, 7WK 8, 7WK9, 7WKA, 7WLC, 7WM0, 7WN2, 7WNB, 7WNM, 7WO4, 7WO5, 7W07, 7WOA, 7WOB, 7W OC, 7WOG, 7WON, 7WOP, 7WOQ, 7WOR, 7WOS, 7WOU, 7WOV, 7WOW, 7WP0, 7WP1, 7WP2, 7 WP5, 7WP6, 7WP8, 7WP9, 7WPA, 7WPB, 7WPC, 7WPD, 7WPE, 7WPF, 7 WPH, 7WQV, 7WR8, 7W RH, 7WRI, 7WRJ, 7WRV, 7WS0, 7WS1, 7WS2, 7WS3, 7WS4, 7WS5, 7WS6, 7WS7, 7WS8, 7WS9, 7 WSA, 7WSE, 7WSH, 7WSK, 7WT7, 7WT8, 7WT9, 7WTF, 7WTG, 7WTH, 7WTI, 7WTJ, 7WTK, 7W UE, 7WUH, 7WVL, 7WVP, 7WVQ, 7WWI, 7WWJ, 7WWK, 7WWL, 7WWM, 7WXZ, 7WZ1, 7WZ2, 7X1M, 7X25, 7X2H, 7X2K, 7X2L, 7X2M, 7X63, 7×66, 7×6A, 7X7D, 7X7E, 7X7N, 7X7T, 7X7U, 7X8 W, 7X8Y, 7X8Z, 7X90, 7×91, 7×92, 7X93, 7×94, 7×95, 7X96, 7X9E, 7XA7, 7XAZ, 7XB0, 7XB1, 7XBY, 7XCH, 7XCI, 7XCK, 7XCO, 7XCP, 7XCZ, 7XD2, 7XDA, 7XDB, 7XDK, 7XDL, 7XEG, 7XEI, 7X H8, 7XIC, 7XID, 7XIK, 7XIL, 7XIW, 7XIX, 7XIY, 7XIZ, 7XJ6, 7XJ8, 7XJ9, 7XMX, 7XMZ, 7XNQ, 7X NR, 7XNS, 7X04, 7×05, 7×06, 7X07, 7×08, 7×09, 7XOA, 7XOB, 7XOC, 7XOD, 7XRP, 7XS8, 7X SA, 7XSB, 7XSC, 7XST, 7XTZ, 7XU0, 7XU1, 7XU2, 7XU3, 7XU4, 7XU5, 7XU6, 7XWA, 7XXL, 7Y0 C, 7Y0V, 7Y1Y, 7Y1Z, 7Y42, 7Y6D, 7Y6K, 7Y6L, 7Y6N, 7Y75, 7Y76, 7Y7J, 7Y7K, 7Y8J, 7Y9N, 7Y9 S, 7Y9Z, 7YA0, 7YA1, 7YAD, 7YBI, 7YBJ, 7YC5, 7YCK, 7YCL, 7YCN, 7YCO, 7YD1, 7YDI, 7YDY, 7YE5, 7YE9, 7YEG, 7YH6, 7YH7, 7YHW, 7YJ3, 7YKJ, 7YOW, 7YQT, 7YQU, 7YQV, 7YQW, 7YQ X, 7YQY, 7YQZ, 7YR0, 7YR1, 7YR2, 7YR3, 7YTN, 7YUE, 7YV8, 7YVE, 7YVF, 7YVG, 7YVH, 7YV 1, 7YVJ, 7YVK, 7YVL, 7YVM, 7YVN, 7YVO, 7YVP, 7YVU, 7Z0X, 7Z0Y, 7ZIA, 7Z1B, 7ZIC, 7ZID, 7ZIE, 7Z3Z, 7Z6V, 7Z7X, 7Z85, 7Z86, 7Z80, 7Z9Q, 7Z9R, 7ZBU, 7ZCE, 7ZCF, 7ZDQ, 7ZF3, 7ZF4, 7 ZF5, 7ZF7, 7ZF8, 7ZF9, 7ZFA, 7ZFB, 7ZFC, 7ZFD, 7ZFE, 7ZJL, 7ZR2, 7ZR7, 7ZR8, 7ZR9, 7ZRC, 7Z RV, 7ZSD, 7ZSS, 7ZXU, 8A99, 8AAA, 8AQS, 8AQT, 8AQU, 8AQV, 8AQW, 8BBN, 8BBO, 8BCZ, 8B E1, 8BEV, 8BGG, 8BH5, 8BON, 8BSE, 8BSF, 8CIV, 8C3V, 8C8P, 8CIM, 8CSA, 8CSJ, 8CWI, 8CWK, 8CWU, 8CWV, 8CXN, 8CXQ, 8CY6, 8CY7, 8CY9, 8CYA, 8CYB, 8CYC, 8CYD, 8CYJ, 8CZI, 8DOZ, 8D36, 8D47, 8D48, 8D55, 8D56, 8D5A, 8D6Z, 8D8Q, 8D8R, 8DAD, 8DAO, 8DCC, 8DCE, 8DF5, 8DG U, 8DI5, 8DLI, 8DLJ, 8DLK, 8DLL, 8DLM, 8DLN, 8DLO, 8DLP, 8DLQ, 8DLR, 8DLS, 8DLT, 8DLU, 8DLV, 8DLW, 8DLX, 8DLY, 8DLZ, 8DM0, 8DM1, 8DM2, 8DM3, 8DM4, 8DM5, 8DM6, 8DM7, 8D M8, 8DM9, 8DMA, 8DNN, 8DT3, 8DT8, 8DTR, 8DTT, 8DTX, 8DV1, 8DV2, 8DW2, 8DW3, 8DW9, 8 DWA, 8DXS, 8DXT, 8DXU, 8DYA, 8DZH, 8DZI, 8E1G, 8EL2, 8ELH, 8ELJ, 8ELO, 8ELP, 8ELQ, 8E O0, 8EPN, 8EPP, 8EPQ, 8ERQ, 8ERR, 8F0G, 8F0H, 8FA1, 8FA2, 8FEZ, 8FU7, 8FU8, 8FU9, 8GB0, 8G B5, 8GB6, 8GB7, 8GB8, 8GJM, 8GJN, 8GOM, 8GON, 8GOU, 8GPY, 8GRY, 8GS6, 8GS9, 8GTO, 8GT P, 8GTQ, 8GX9, 8GZ5, 8GZZ, 8H00, 8H01, 8H06, 8H07, 8H08, 8H3D, 8H3E, 8H3M, 8H3N, 8H5C, 8H C2, 8HC3, 8HC4, 8HC5, 8HC6, 8HC7, 8HC8, 8HC9, 8HCA, 8HCB, 8HEB, 8HEC, 8HED, 8HHX, 8HH Y, 8HHZ, 8HN6, 8HN7, 815H, 8151, 8IDN, 8IF2, 8IOS, 8IOT, 8IOU, 8IOV, 8ITU, 8JIQ, 8J26, or 8SMT. Each of these PDB IDs is incorporated by reference herein in its entirety. The amino acid sequence of the SARS-COV-2 S glycoprotein associated with each entry may be accessed using FASTA.

In embodiments, the SARS-COV-2 S glycoprotein is a wild-type SARS-COV-2 S glycoprotein, or a SARS-COV-2 S glycoprotein from a SARS-COV-2 variant thereof. In embodiments, the variant of SARS-COV-2 is a B.1.1.7 SARS-COV-2 strain; a B.1.351 SARS-CoV-2 strain; a P.1 SARS-COV-2 strain; a Cal.20C SARS-COV-2 strain; a B.1.617.2 SARS-COV-2 strain; a B.1.525 SARS-COV-2 strain; a B.1.526 SARS-COV-2 strain; a B.1.617.1 SARS-COV-2 strain; a C.37 SARS-CoV-2 strain; a B.1.621 SARS-CoV-2 strain; or a B.1.1.529 SARS-CoV-2 strain. In embodiments, the SARS-CoV-2 S glycoprotein contains a transmembrane domain.

The wild-type SARS-CoV-2 S glycoprotein contains a furin cleavage site, RRAR (SEQ ID NO: 6) at positions 669-672 of the SARS-CoV-2 S glycoprotein of SEQ ID NO: 2. In embodiments, the SARS-CoV-2 S glycoprotein has an inactive furin cleavage site. In embodiments, the inactive furin cleavage site has the amino acid sequence of any one of SEQ ID NOS: 7-34, 97, and 111. In embodiments, the amino acid sequence of the inactive furin cleavage site is GG.

In embodiments, the amino acid sequence of the inactive furin cleavage site is QQAQ (SEQ ID NO: 7).

In embodiments, one or more of the amino acids comprising the native furin cleavage site is mutated to any natural amino acid. In embodiments, one or more of the amino acids comprising the native furin cleavage site is deleted.

In embodiments, one or more of the amino acids comprising the native furin cleavage site is mutated to glutamine. In embodiments, 1, 2, 3, or 4 amino acids may be mutated to glutamine. In embodiments, one of the arginines comprising the native furin cleavage site is mutated to glutamine. In embodiments, two of the arginines comprising the native furin cleavage site are mutated to glutamine. In embodiments, three of the arginines comprising the native furin cleavage site are mutated to glutamine.

In embodiments, one or more of the amino acids comprising the native furin cleavage site, is mutated to alanine. In embodiments, 1, 2, 3, or 4 amino acids may be mutated to alanine. embodiments, one of the arginines comprising the native furin cleavage site is mutated to alanine. In embodiments, two of the arginines comprising the native furin cleavage site are mutated to alanine. In embodiments, three of the arginines comprising the native furin cleavage site are mutated to alanine.

In embodiments, one or more of the amino acids comprising the native furin cleavage site is mutated to glycine. In embodiments, 1, 2, 3, or 4 amino acids may be mutated to glycine. In embodiments, one of the arginines of the native furin cleavage site is mutated to glycine. In embodiments, two of the arginines comprising the native furin cleavage site are mutated to glycine. In embodiments, three of the arginines comprising the native furin cleavage site are mutated to glycine.

In embodiments, one or more of the amino acids comprising the native furin cleavage site, is mutated to asparagine. For example 1, 2, 3, or 4 amino acids may be mutated to asparagine. In embodiments, one of the arginines comprising the native furin cleavage site is mutated to asparagine. In embodiments, two of the arginines comprising the native furin cleavage site are mutated to asparagine. In embodiments, three of the arginines comprising the native furin cleavage site are mutated to asparagine.

In embodiments, the active furin cleavage site (SEQ ID NO: 67) of the SARS-CoV-2 S glycoproteins described herein is replaced with an inactivated furin cleavage site of the table below.

### Inactivated Furin Cleavage Sites

| Amino Acid Sequence of Furin | Active or Inactive Furin |
|---|---|
| Cleavage Site | Cleavage Site |
| RRAR (SEQ ID NO: 6) | Active |
| QQAQ (SEQ ID NO: 7) | Inactive |
| QRAR (SEQ ID NO: 8) | Inactive |
| RQAR (SEQ ID NO: 9) | Inactive |
| RRAQ (SEQ ID NO: 10) | Inactive |
| QQAR (SEQ ID NO: 11) | Inactive |
| RQAQ (SEQ ID NO: 12) | Inactive |
| QRAQ (SEQ ID NO: 13) | Inactive |
| NNAN (SEQ ID NO: 14) | Inactive |
| NRAR (SEQ ID NO: 15) | Inactive |
| RNAR (SEQ ID NO: 16) | Inactive |
| RRAN (SEQ ID NO: 17) | Inactive |
| NNAR (SEQ ID NO: 18) | Inactive |
| RNAN (SEQ ID NO: 19) | Inactive |
| NRAN (SEQ ID NO: 20) | Inactive |
| AAAA (SEQ ID NO: 21) | Inactive |
| ARAR (SEQ ID NO: 22) | Inactive |
| RAAR (SEQ ID NO: 23) | Inactive |
| RRAA (SEQ ID NO: 24) | Inactive |
| AAAR (SEQ ID NO: 25) | Inactive |
| RAAA (SEQ ID NO: 26) | Inactive |
| ARAA (SEQ ID NO: 27) | Inactive |
| GGAG (SEQ ID NO: 28) | Inactive |
| GRAR (SEQ ID NO: 29) | Inactive |
| RGAR (SEQ ID NO: 30) | Inactive |
| RRAG (SEQ ID NO: 31) | Inactive |
| GGAR (SEQ ID NO: 32) | Inactive |
| RGAG (SEQ ID NO: 33) | Inactive |
| GRAG (SEQ ID NO: 34) | Inactive |
| GSAS (SEQ ID NO: 97) | Inactive |
| GSGA (SEQ ID NO: 111) | Inactive |

In embodiments, the SARS-CoV-2 S glycoproteins contain a mutation at Lys-973 of the native SARS-CoV-2 S glycoprotein (SEQ ID NO: 2). In embodiments, Lys-973 is mutated to any natural amino acid. In embodiments, Lys-973 is mutated to proline. In embodiments, Lys-973 is mutated to glycine.

In embodiments, the SARS-CoV-2 S glycoproteins contain a mutation at Val-974 of the native SARS-CoV-2 S glycoprotein (SEQ ID NO: 2). In embodiments, Val-974 is mutated to any natural amino acid, as compared to the SARS-CoV-2 S glycoprotein of SEQ ID NO: 2. In embodiments, Val-974 is mutated to proline. In embodiments, Val-974 is mutated to glycine, as compared to the SARS-CoV-2 S glycoprotein of SEQ ID NO: 2.

In embodiments, the SARS-CoV-2 S glycoproteins contain a mutation at Lys-973 and Val-974 of the native CoV Spike (S) polypeptide (SEQ ID NO: 2). In embodiments, Lys-973 and Val-974 are mutated to any natural amino acid, as compared to the SARS-CoV-2 S glycoprotein of SEQ ID NO: 2. In embodiments, Lys-973 and Val-974 are mutated to proline, as compared to the SARS-CoV-2 S glycoprotein of SEQ ID NO: 2. An exemplary SARS-CoV-2 S glycoprotein that contains proline at positions 973 and 974 is the glycoprotein of SEQ ID NO: 87, wherein the SARS-CoV-2 S glycoprotein is numbered according to SEQ ID NO: 2.

In embodiments, the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 2, 4, 38, 41, 44, 48, 51, 54, 58, 61, 63, 65, 67, 73, 75, 78, 79, 82, 83, 85, 87, 89, 106, 110, 132, 133, 114, 138, 141, 144, 147, 151, 153, 156, 158, 174, 175, 176, 181-184, 186, 188, 190, 195, 217-228 233-236, 243, 255-264, 273-280, 283-284, 287-288, 291-292, 294, 296, 298, 300, 302, 304, 306, 308, 310, 312, 314, 316, 318, 320, 322, 324, 236, 328, 329, 330, 331, 332 and 333.

Providing a Surface Coated with a SARS-CoV-2 S Glycoprotein

In embodiments, the methods described herein require providing a surface coated with a SARS-CoV-2 S glycoprotein. Numerous examples of SARS-CoV-2 S glycoproteins are provided herein. In embodiments, the surface is a chip or a microplate. In embodiments, the microplate comprises polystyrene. In embodiments, the microplate is 96-well or 384-well polystyrene plate. In embodiments, the surface is a microplate that is filled with a solution.

### Exposing the Surface to a Biological Sample

In embodiments, the surface is exposed to a biological sample. In embodiments, the surface is exposed to the biological sample for from 10 minutes to about 72 hours. In embodiments, the surface is exposed to the biological sample for about 10 minutes, about 15 minutes, about 20 minutes, about 25 minutes, about 30 minutes, about 35 minutes, about 40 minutes, about 45 minutes, about 50 minutes, about 55 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 49 hours, about 50 hours, about 51 hours, about 52 hours, about 53 hours, about 54 hours, about 55 hours, about 56 hours, about 57 hours, about 58 hours, about 59 hours, about 60 hours, about 61 hours, about 62 hours, about 63 hours, about 64 hours, about 65 hours, about 66 hours, about 67 hours, about 68 hours, about 69 hours, about 70 hours, about 71 hours, or about 72 hours, including any range or any value therebetween. In embodiments, the surface is exposed to the biological sample for about 1 hour or 2 hours. In embodiments, the surface is exposed to the biological sample for about 2 hours. In embodiments, the surface is exposed to the biological sample at a temperature from 2-8° C. or from 8-37° C. In embodiments, the surface is exposed to the biological sample at a temperature of 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., or about 37° C.

In embodiments, the biological sample is saliva, a nasopharyngeal swab, sputum, saliva, urine, a fecal sample, cerebrospinal fluid, synovial fluid, serum, blood, or plasma. In embodiments, the biological sample is from a patient that has previously had COVID-19. In embodiments, the biological sample is from a patient that has been administered an immunogenic composition against a SARS-CoV-2 virus or a variant thereof.

### Exposing the Surface to a Secondary Antibody

In embodiments, the surface is exposed to a secondary antibody. In embodiments, the secondary antibody is an anti-human IgG4 antibody. An anti-human IgG4 antibody binds to IgG4 antibodies. In embodiments, the secondary antibody is an anti-human IgG1 antibody. An anti-human IgG1 antibody binds to IgG1 antibodies. In embodiments, the secondary antibody is an anti-human IgG antibody. An anti-human IgG antibody binds to all IgG.

In embodiments, the secondary antibody is attached to a tag. In embodiments, the secondary antibody is covalently attached to a tag. In embodiments, the secondary antibody is non-covalently attached to a tag. In embodiments, the tag is a His tag. In embodiments, the tag contains an epitope. For example, the tag may be a polyglutamate tag, a FLAG-tag, a HA-tag, a polyHis-tag (having about 5-10 histidines) (SEQ ID NO: 101), a hexahistidine tag (SEQ ID NO: 100), an 8X-His-tag (having eight histidines) (SEQ ID NO: 102), a Myc-tag, a Glutathione-S-transferase-tag, a Green fluorescent protein-tag, Maltose binding protein-tag, a Thioredoxin-tag, or an Fc-tag. In embodiments, the tag is a protease cleavage site. Non-limiting examples of protease cleavage sites include the HRV3C protease cleavage site, chymotrypsin, trypsin, elastase, endopeptidase, caspase-1, caspase-2, caspase-3, caspase-4, caspase-5, caspase-6, caspase-7, caspase-8, caspase-9, caspase-10, enterokinase, factor Xa, Granzyme B, TEV protease, and thrombin. In embodiments, the protease cleavage site is an HRV3C protease cleavage site. In embodiments, the tag is horseradish peroxidase (HRP).

In embodiments, the surface is exposed to secondary antibody for from 10 minutes to about 72 hours. In embodiments, the surface is exposed to secondary antibody for about 10 minutes, about 15 minutes, about 20 minutes, about 25 minutes, about 30 minutes, about 35 minutes, about 40 minutes, about 45 minutes, about 50 minutes, about 55 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 49 hours, about 50 hours, about 51 hours, about 52 hours, about 53 hours, about 54 hours, about 55 hours, about 56 hours, about 57 hours, about 58 hours, about 59 hours, about 60 hours, about 61 hours, about 62 hours, about 63 hours, about 64 hours, about 65 hours, about 66 hours, about 67 hours, about 68 hours, about 69 hours, about 70 hours, about 71 hours, or about 72 hours, including any range or any value therebetween. In embodiments, the surface is exposed to secondary antibody for about 1 hour.

Detecting Anti-SARS-CoV-2 IgG1, IgG4, and Total IgG that is Bound to the Surface

In embodiments, the methods comprise detecting the anti-SARS-CoV-2 IgG1, IgG4, and total IgG that is bound to the surface.

In embodiments, detecting anti-SARS-CoV-2 IgG1 comprises (i) contacting the surface with a secondary antibody that binds to IgG1 antibodies, wherein the antibody is tagged with horseradish peroxidase; and (ii) contacting the surface with 3,3′,5,5′-tetramethylbenzidine substrate. In embodiments, detecting anti-SARS-CoV-2 IgG4 comprises (i) contacting the surface with a secondary antibody that binds to IgG4 antibodies, wherein the antibody is tagged with horseradish peroxidase; and (ii) contacting the surface with 3,3′,5,5′-tetramethylbenzidine substrate. In embodiments, detecting anti-SARS-CoV-2 IgG comprises (i) contacting the surface with a secondary antibody that binds to IgG antibodies, wherein the antibody is tagged with horseradish peroxidase; and (ii) contacting the surface with 3,3′,5,5′-tetramethylbenzidine substrate. In embodiments, detecting comprises determining the absorbance of the surface. In embodiments, detecting comprises determining the absorbance of the surface at a wavelength from 400 nm to about 650 nm. In embodiments, detecting comprises determining the absorbance of the surface at a wavelength of about 400 nm, about 410 nm, about 420 nm, about 430 nm, about 440 nm, about 450 nm, about 460 nm, about 470 nm, about 480 nm, about 490 nm, about 500 nm, about 510 nm, about 520 nm, about 530 nm, about 540 nm, about 550 nm, about 560 nm, about 570 nm, about 580 nm, about 590 nm, about 600 nm, about 610 nm, about 620 nm, about 630 nm, about 640 nm, or about 650 nm, including all values and ranges therebetween. In embodiments, detecting comprises determining the absorbance of the surface at a wavelength of about 450 nm. In embodiments, the surface is contacted with 3,3′,5,5′-tetramethylbenzidine substrate for about 5 minutes to about 1 hour. In embodiments, the surface is contacted with 3,3′,5,5′-tetramethylbenzidine substrate for about 20 minutes.

In embodiments, the methods used herein can be used to evaluate the immunogenicity of compositions and vaccine compositions against SARS-CoV-2. In embodiments, the immunogenic compositions and vaccine compositions target a SARS-CoV-2 virus, or a heterogeneous SARS-CoV-2 strain. In embodiments, the immunogenic composition or vaccine composition comprises a SARS-CoV-2 S glycoprotein or a nucleic acid (e.g., mRNA) that encodes a SARS-CoV-2 S glycoprotein. In embodiments, the immunogenic composition or vaccine composition comprises a viral vector that expresses a SARS-CoV-2 S glycoprotein. In embodiments, the immunogenic compositions or vaccine compositions comprise a SARS-CoV-2 S glycoprotein that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 87.

In embodiments, the immunogenic compositions or vaccine compositions comprise an adjuvant. Exemplary adjuvants are described below.

### Aluminum-Based Adjuvants

In embodiments, the adjuvant may be alum (e.g. AlPO₄ or Al(OH)₃). Typically, the nanoparticle is substantially bound to the alum. For example, the nanoparticle may be at least 80% bound, at least 85% bound, at least 90% bound or at least 95% bound to the alum. Often, the nanoparticle is 92% to 97% bound to the alum in a composition. The amount of alum is present per dose is typically in a range between about 400 μg to about 1250 μg. For example, the alum may be present in a per dose amount of about 300 μg to about 900 μg, about 400 μg to about 800 μg, about 500 μg to about 700 μg, about 400 μg to about 600 μg, or about 400 μg to about 500 μg. Typically, the alum is present at about 400 μg for a dose of 120 μg of the protein nanoparticle.

### Saponin Adjuvants

Adjuvants containing saponin may also be combined with the immunogens disclosed herein. Saponins are glycosides derived from the bark of the Quillaja saponaria Molina tree. Typically, saponin is prepared using a multi-step purification process resulting in multiple fractions. As used, herein, the term “a saponin fraction from Quillaja saponaria Molina” is used generically to describe a semi-purified or defined saponin fraction of Quillaja saponaria or a substantially pure fraction thereof.

### Saponin Fractions

Several approaches for producing saponin fractions are suitable. Fractions A, B, and C are described in U.S. Pat. No. 6,352,697 and may be prepared as follows. A lipophilic fraction from Quil A, a crude aqueous Quillaja saponaria Molina extract, is separated by chromatography and eluted with 70% acetonitrile in water to recover the lipophilic fraction. This lipophilic fraction is then separated by semi-preparative HPLC with elution using a gradient of from 25% to 60% acetonitrile in acidic water. The fraction referred to herein as “Fraction A” or “QH-A” is, or corresponds to, the fraction, which is eluted at approximately 39% acetonitrile. The fraction referred to herein as “Fraction B” or “QH-B” is, or corresponds to, the fraction, which is eluted at approximately 47% acetonitrile. The fraction referred to herein as “Fraction C” or “QH-C” is, or corresponds to, the fraction, which is eluted at approximately 49% acetonitrile. Additional information regarding purification of Fractions is found in U.S. Pat. No. 5,057,540. When prepared as described herein, Fractions A, B and C of Quillaja saponaria Molina each represent groups or families of chemically closely related molecules with definable properties. The chromatographic conditions under which they are obtained are such that the batch-to-batch reproducibility in terms of elution profile and biological activity is highly consistent.

Other saponin fractions have been described. Fractions B3, B4 and B4b are described in EP 0436620. Fractions QA1-QA22 are described EP03632279 B2, Q-VAC (Nor-Feed, AS Denmark), Quillaja saponaria Molina Spikoside (lsconova AB, Ultunaallén 2B, 756 51 Uppsala, Sweden). Fractions QA-1, QA-2, QA-3, QA-4, QA-5, QA-6, QA-7, QA-8, QA-9, QA-10, QA-11, QA-12, QA-13, QA-14, QA-15, QA-16, QA-17, QA-18, QA-19, QA-20, QA-21, and QA-22 of EP 0 3632 279 B2, especially QA-7, QA-17, QA-18, and QA-21 may be used. They are obtained as described in EP 0 3632 279 B2, especially at page 6 and in Example 1 on page 8 and 9.

The saponin fractions described herein and used for forming adjuvants are often substantially pure fractions; that is, the fractions are substantially free of the presence of contamination from other materials. In particular aspects, a substantially pure saponin fraction may contain up to 40% by weight, up to 30% by weight, up to 25% by weight, up to 20% by weight, up to 15% by weight, up to 10% by weight, up to 7% by weight, up to 5% by weight, up to 2% by weight, up to 1% by weight, up to 0.5% by weight, or up to 0.1% by weight of other compounds such as other saponins or other adjuvant materials.

### ISCOM Structures

Saponin fractions may be administered in the form of a cage-like particle referred to as an ISCOM (Immune Stimulating COMplex). ISCOMs may be prepared as described in EP0109942B1, EP0242380B1 and EP0180546 B1. In particular embodiments a transport and/or a passenger antigen may be used, as described in EP 9600647-3 (PCT/SE97/00289).

### Matrix Adjuvants

In embodiments, the ISCOM is an ISCOM matrix complex. An ISCOM matrix complex comprises at least one saponin fraction and a lipid. The lipid is at least a sterol, such as cholesterol. In particular aspects, the ISCOM matrix complex also contains a phospholipid. The ISCOM matrix complexes may also contain one or more other immunomodulatory (adjuvant-active) substances, not necessarily a glycoside, and may be produced as described in EP0436620B1, which is incorporated by reference in its entirety herein.

In other aspects, the ISCOM is an ISCOM complex. An ISCOM complex contains at least one saponin, at least one lipid, and at least one kind of antigen or epitope. The ISCOM complex contains antigen associated by detergent treatment such that that a portion of the antigen integrates into the particle. In contrast, ISCOM matrix is formulated as an admixture with antigen and the association between ISCOM matrix particles and antigen is mediated by electrostatic and/or hydrophobic interactions.

According to one embodiment, the saponin fraction integrated into an ISCOM matrix complex or an ISCOM complex, or at least one additional adjuvant, which also is integrated into the ISCOM or ISCOM matrix complex or mixed therewith, is selected from fraction A, fraction B, or fraction C of Quillaja saponaria, a semipurified preparation of Quillaja saponaria, a purified preparation of Quillaja saponaria, or any purified sub-fraction e.g., QA 1-21.

In particular aspects, each ISCOM particle may contain at least two saponin fractions. Any combinations of weight % of different saponin fractions may be used. Any combination of weight % of any two fractions may be used. For example, the particle may contain any weight % of fraction A and any weight % of another saponin fraction, such as a crude saponin fraction or fraction C, respectively. Accordingly, in particular aspects, each ISCOM matrix particle or each ISCOM complex particle may contain from 0.1 to 99.9 by weight, 5 to 95% by weight, 10 to 90% by weight 15 to 85% by weight, 20 to 80% by weight, 25 to 75% by weight, 30 to 70% by weight, 35 to 65% by weight, 40 to 60% by weight, 45 to 55% by weight, 40 to 60% by weight, or 50% by weight of one saponin fraction, e.g. fraction A and the rest up to 100% in each case of another saponin e.g. any crude fraction or any other faction e.g. fraction C. The weight is calculated as the total weight of the saponin fractions. Examples of ISCOM matrix complex and ISCOM complex adjuvants are disclosed in U.S Published Application No. 2013/0129770, which is incorporated by reference in its entirety herein.

In particular embodiments, the ISCOM matrix or ISCOM complex comprises from 5-99% by weight of one fraction, e.g. fraction A and the rest up to 100% of weight of another fraction e.g. a crude saponin fraction or fraction C. The weight is calculated as the total weight of the saponin fractions.

In another embodiment, the ISCOM matrix or ISCOM complex comprises from 40% to 99% by weight of one fraction, e.g. fraction A and from 1% to 60% by weight of another fraction, e.g. a crude saponin fraction or fraction C. The weight is calculated as the total weight of the saponin fractions.

In yet another embodiment, the ISCOM matrix or ISCOM complex comprises from 70% to 95% by weight of one fraction e.g., fraction A, and from 30% to 5% by weight of another fraction, e.g., a crude saponin fraction, or fraction C. The weight is calculated as the total weight of the saponin fractions. In other embodiments, the saponin fraction from Quillaja saponaria Molina is selected from any one of QA 1-21.

In addition to particles containing mixtures of saponin fractions, ISCOM matrix particles and ISCOM complex particles may each be formed using only one saponin fraction. Compositions disclosed herein may contain multiple particles wherein each particle contains only one saponin fraction. That is, certain compositions may contain one or more different types of ISCOM-matrix complexes particles and/or one or more different types of ISCOM complexes particles, where each individual particle contains one saponin fraction from Quillaja saponaria Molina, wherein the saponin fraction in one complex is different from the saponin fraction in the other complex particles.

In particular aspects, one type of saponin fraction or a crude saponin fraction may be integrated into one ISCOM matrix complex or particle and another type of substantially pure saponin fraction, or a crude saponin fraction, may be integrated into another ISCOM matrix complex or particle. A composition or vaccine may comprise at least two types of complexes or particles each type having one type of saponins integrated into physically different particles.

In the compositions, mixtures of ISCOM matrix complex particles and/or ISCOM complex particles may be used in which one saponin fraction Quillaja saponaria Molina and another saponin fraction Quillaja saponaria Molina are separately incorporated into different ISCOM matrix complex particles and/or ISCOM complex particles.

The ISCOM matrix or ISCOM complex particles, which each have one saponin fraction, may be present in composition at any combination of weight %. In particular aspects, a composition may contain 0.1% to 99.9% by weight, 5% to 95% by weight, 10% to 90% by weight, 15% to 85% by weight, 20% to 80% by weight, 25% to 75% by weight, 30% to 70% by weight, 35% to 65% by weight, 40% to 60% by weight, 45% to 55% by weight, 40 to 60% by weight, or 50% by weight, of an ISCOM matrix or complex containing a first saponin fraction with the remaining portion made up by an ISCOM matrix or complex containing a different saponin fraction. In aspects, the remaining portion is one or more ISCOM matrix or complexes where each matrix or complex particle contains only one saponin fraction. In other aspects, the ISCOM matrix or complex particles may contain more than one saponin fraction.

In particular compositions, the only saponin fraction in a first ISCOM matrix or ISCOM complex particle is Fraction A and the only saponin fraction in a second ISCOM matrix or ISCOM complex particle is Fraction C.

In embodiments, the Fraction A of Quillaja saponaria Molina accounts for at least about 80%, 81%, 82%, 83%, 84%, 85%, 86%, 87%, 88%, 89%, 90%, 91%, 92%, 93%, 94%, 95%, 96%, 97%, 98%, or 99% by weight, and fraction C of Quillaja saponaria Molina accounts for the remainder, respectively, of the sum of the weights of fraction A of Quillaja saponaria Molina and fraction C of Quillaja saponaria Molina in the adjuvant.

Preferred compositions comprise a first ISCOM matrix containing Fraction A and a second ISCOM matrix containing Fraction C, wherein the Fraction A ISCOM matrix constitutes about 70% per weight of the total saponin adjuvant, and the Fraction C ISCOM matrix constitutes about 30% per weight of the total saponin adjuvant. In another preferred composition, the Fraction A ISCOM matrix constitutes about 85% per weight of the total saponin adjuvant, and the Fraction C ISCOM matrix constitutes about 15% per weight of the total saponin adjuvant. In another preferred composition, the Fraction A ISCOM matrix constitutes about 92% per weight of the total saponin adjuvant, and the Fraction C ISCOM matrix constitutes about 8% per weight of the total saponin adjuvant. Thus, in certain compositions, the Fraction A ISCOM matrix is present in a range of about 70% to about 85%, and Fraction C ISCOM matrix is present in a range of about 15% to about 30%, of the total weight amount of saponin adjuvant in the composition. In certain compositions, the Fraction A ISCOM matrix is present in a range of about 70% to about 92%, and Fraction C ISCOM matrix is present in a range of about 8% to about 30%, of the total weight amount of saponin adjuvant in the composition. In embodiments, the Fraction A ISCOM matrix accounts for 50-96% by weight and Fraction C ISCOM matrix accounts for the remainder, respectively, of the sums of the weights of Fraction A ISCOM matrix and Fraction C ISCOM in the adjuvant. In a particularly preferred composition, referred to herein as MATRIX-M™, the Fraction A ISCOM matrix is present at about 85% and Fraction C ISCOM matrix is present at about 15% of the total weight amount of saponin adjuvant in the composition. MATRIX-M™ may be referred to interchangeably as Matrix-M1.

Exemplary QS-7 and QS-21 fractions, their production and their use is described in U.S. Pat. Nos. 5,057,540; 6,231,859; 6,352,697; 6,524,584; 6,846,489; 7,776,343, and 8,173,141, which are incorporated by reference herein.

In embodiments, other adjuvants may be used in addition or as an alternative. The inclusion of any adjuvant described in Vogel et al., “A Compendium of Vaccine Adjuvants and Excipients (2nd Edition),” herein incorporated by reference in its entirety for all purposes, is envisioned within the scope of this disclosure. Other adjuvants include complete Freund's adjuvant (a non-specific stimulator of the immune response containing killed Mycobacterium tuberculosis), incomplete Freund's adjuvants and aluminum hydroxide adjuvant. Other adjuvants comprise GMCSP, BCG, MDP compounds, such as thur-MDP and nor-MDP, CGP (MTP-PE), lipid A, and monophosphoryl lipid A (MPL), MF-59, RIBI, which contains three components extracted from bacteria, MPL, trehalose dimycolate (TDM) and cell wall skeleton (CWS) in a 2% squalene/TWEEN® polysorbate 80 emulsion. In embodiments, the adjuvant may be a paucilamellar lipid vesicle; for example, NOVASOMES®. NOVASOMES® are paucilamellar nonphospholipid vesicles ranging from about 100 nm to about 500 nm. They comprise BRIJ® alcohol ethoxylate 72, cholesterol, oleic acid and squalene. NOVASOMES® have been shown to be an effective adjuvant (see, U.S. Pat. Nos. 5,629,021, 6,387,373, and 4,911,928.

### Example 1: Assays to Measure Anti-rS Proteins IgG1, IgG2, IgG3, and IgG4, and Total Anti-rS IgG in Serum of Patients Administered COVID-19 Vaccines or Previously Infected with SARS-CoV-2

Purpose: The anti-rS proteins IgG1, IgG2, IgG3, and IgG4, and total anti-rS IgG assays were developed to measure the levels of anti-Spike IgG (% of Total anti-Spike IgG) in serum samples from subjects repeatedly immunized with COVID-19 vaccines. These assays allowed for the identification of different IgG subtypes to guide vaccine platform selection. Surrogate ADCP (FcγRIIa binding), surrogate ADCC (FcγRIIIa binding), and ADCD (C1q binding) were also measured.

Samples: Samples were collected: (1) from patients that received three doses of a first vaccine that contained mRNA encoding a SARS-CoV-2 Spike protein before and after the patients were administered a booster of a vaccine containing a SARS-CoV-2 Spike glycoprotein (referred to as “Heterologous 1”); (2) from patients that received three doses of a second vaccine that contained mRNA encoding a SARS-CoV-2 Spike protein before and after the patients were administered a booster of a vaccine containing a SARS-CoV-2 Spike glycoprotein (referred to as “Heterologous 2”); and (3) from patients that received two doses of a vaccine containing a SARS-CoV-2 Spike glycoprotein before and after the patients were administered a booster of the same vaccine (referred to as “Homologous”). The SARS-CoV-2 Spike glycoprotein of the vaccine from (3) and the booster of (1), (2), and (3) contains proline at positions 973 and 974 and an inactive primary furin cleavage site, wherein the SARS-CoV-2 S glycoprotein is numbered according to the polypeptide of SEQ ID NO: 2. The vaccine of (3) and the booster of (1), (2), and (3) further contains a saponin adjuvant. The saponin adjuvant contained two iscom particles, wherein: the first iscom particle comprises fraction A of Quillaja saponaria Molina and not fraction C of Quillaja saponaria Molina; and the second iscom particle comprises fraction C of Quillaja saponaria Molina and not fraction A of Quillaja saponaria Molina. Fraction A and Fraction C account for 85% and 15% by weight, respectively, of the sum of the weights of fraction A of Quillaja saponaria Molina and fraction C of Quillaja saponaria Molina in the adjuvant.

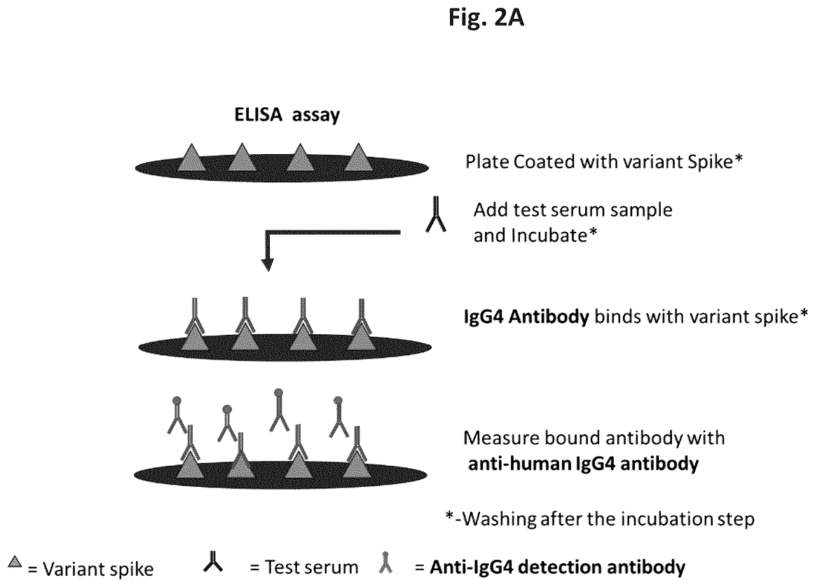

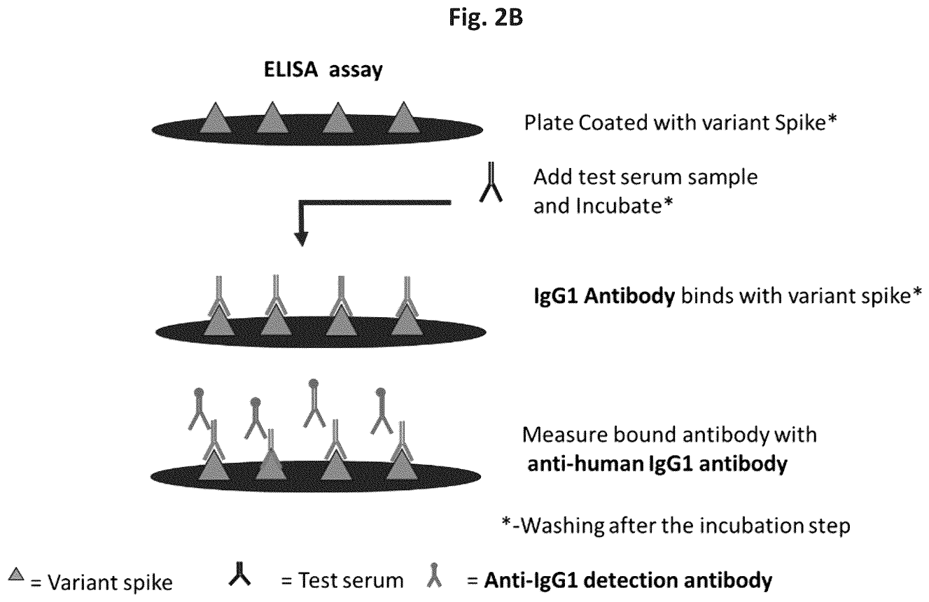

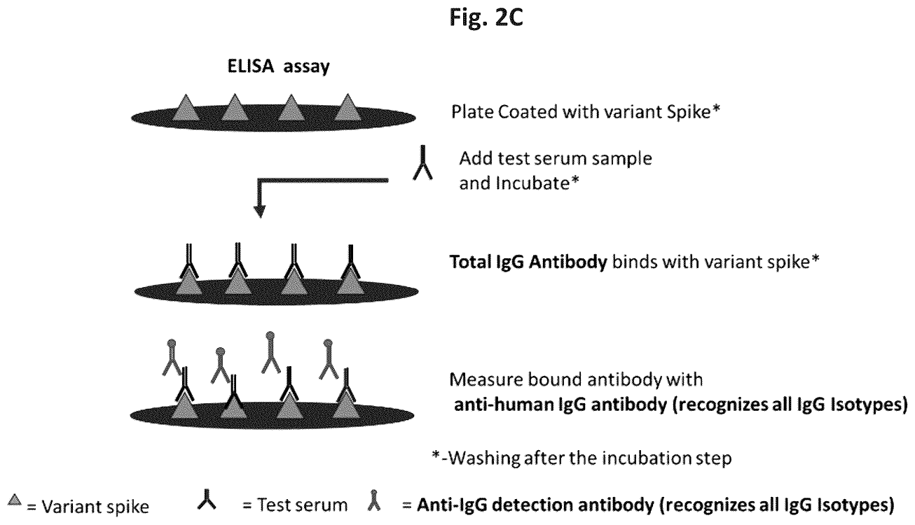

Each sample was subjected to an anti-Spike IgG4 assay (FIG. 2A), an anti-Spike IgG1 assay (FIG. 2B), and a total anti-Spike IgG assay (FIG. 2C), as described below and illustrated in FIGS. 2A-2C. The SARS-CoV-2 S glycoprotein utilized in the assay (also referred to as “SARS-CoV-2 rS protein”) contains proline at positions 973 and 974 and an inactive primary furin cleavage site, wherein the SARS-CoV-2 S glycoprotein is numbered according to the polypeptide of SEQ ID NO: 2.

Anti-Spike IgG4 assay: Assay plates (Thermo Fisher Scientific, Waltham, MA, USA) were coated with 1-2 μg/mL SARS-CoV-2 rS protein (produced at Novavax, Inc., Gaithersburg, MD, USA) for 15 to 72 hours at 2-8° C. Plates were then washed with phosphate-buffered saline with Tween 20 (PBST) and blocked for 1 hour with blocking buffer (Thermo Fisher Scientific). Human serum samples (reference standards, quality controls, or test sera) were then added to the wells, allowing anti-rS protein IgG antibodies to bind to rS protein coated on plate (2 hours of incubation). An anti-SARS-CoV-2 Spike RBD IgG4 monoclonal antibody (Acro Biosystems, Cat #SPD-M402a) was used as a reference standard to measure the IgG4 antibodies in serum samples. The plates were again washed with PBST, and then a mouse anti-human IgG4 secondary antibody conjugated with horseradish peroxidase (HRP; from Invitrogen) was added and incubated for 1 hour at room temperature. A final wash step was performed, followed by addition of 3,3′5,5′-tetramethylbenzidine substrate (TMB, from Thermo Fisher Scientific). The reaction was stopped after 20 minutes by TMB stop solution (Scytek Laboratories, Logan, UT, USA). The optical density (OD) of the chromogenic signal is directly proportional to the amount of anti-rS IgG4 captured on the plate, providing a quantifiable measurement of IgG4 concentration in the serum sample. Anti-Spike IgG4 concentration was calculated by interpolating to the levels of the IgG4 reference standard curve.

Anti-Spike IgG1 assay: Assay plates (Thermo Fisher Scientific, Waltham, MA, USA) were coated with 1-2 μg/mL SARS-CoV-2 rS protein (produced at Novavax, Inc., Gaithersburg, MD, USA) for 15 to 72 hours at 2-8° C. Plates were then washed with phosphate-buffered saline with Tween 20 (PBST) and blocked for 1 hour with blocking buffer (Thermo Fisher Scientific). Human serum samples (reference standards, quality controls, or test sera) were then added to the wells, allowing anti-rS protein IgG antibodies to bind to S protein coated on plate (2 hours of incubation). An anti-SARS-CoV-2 Spike RBD IgG1 monoclonal antibody (Acro Biosystems, Cat #SPD-M265) was used as a reference standard. The plates were again washed with PBST, and then a mouse anti-human IgG1 secondary antibody conjugated with horseradish peroxidase (HRP; from Sino Biological) was added and incubated for 1 hour at room temperature. A final wash step was performed, followed by addition of 3,3′5,5′-tetramethylbenzidine substrate (TMB, from Thermo Fisher Scientific). The reaction was stopped after 25 minutes by TMB stop solution (Scytek Laboratories, Logan, UT, USA). The optical density (OD) of the chromogenic signal is directly proportional to the amount of anti-rS IgG1 captured on the plate, providing a quantifiable measurement of IgG1 concentration in the serum sample. Anti-Spike IgG1 concentration was calculated by interpolating to the levels of the IgG1 reference standard curve.

Anti-Spike IgG2 assay: Assay plates (Thermo Fisher Scientific, Waltham, MA, USA) were coated with 1-2 μg/mL SARS-CoV-2 rS protein (produced at Novavax, Inc., Gaithersburg, MD, USA) for 15 to 72 hours at 2-8° C. Plates were then washed with phosphate-buffered saline with Tween 20 (PBST) and blocked for 1 hour with blocking buffer (Thermo Fisher Scientific). Human serum samples (reference standards, quality controls, or test sera) were then added to the wells, allowing anti-rS protein IgG antibodies to bind to S protein coated on plate (2 hours of incubation). An anti-SARS-CoV-2 Spike RBD IgG1 monoclonal antibody (Acro Biosystems, Cat #SPD-M265) was used as a reference standard. The plates were again washed with PBST, and then a mouse anti-human IgG2 secondary antibody conjugated with horseradish peroxidase (HRP; from Sino Biological) was added and incubated for 1 hour at room temperature. A final wash step was performed, followed by addition of 3,3′5,5′-tetramethylbenzidine substrate (TMB, from Thermo Fisher Scientific). The reaction was stopped after 25 minutes by TMB stop solution (Scytek Laboratories, Logan, UT, USA). The optical density (OD) of the chromogenic signal is directly proportional to the amount of anti-rS IgG2 captured on the plate, providing a quantifiable measurement of IgG2 concentration in the serum sample. Anti-Spike IgG2 concentration was calculated by interpolating to the levels of the IgG2 reference standard curve.

Anti-Spike IgG3 assay: Assay plates (Thermo Fisher Scientific, Waltham, MA, USA) were coated with 1-2 μg/mL SARS-CoV-2 rS protein (produced at Novavax, Inc., Gaithersburg, MD, USA) for 15 to 72 hours at 2-8° C. Plates were then washed with phosphate-buffered saline with Tween 20 (PBST) and blocked for 1 hour with blocking buffer (Thermo Fisher Scientific). Human serum samples (reference standards, quality controls, or test sera) were then added to the wells, allowing anti-rS protein IgG antibodies to bind to S protein coated on plate (2 hours of incubation). An anti-SARS-CoV-2 Spike RBD IgG3 monoclonal antibody (Acro Biosystems, Cat #SPD-M265) was used as a reference standard. The plates were again washed with PBST, and then a mouse anti-human IgG3 secondary antibody conjugated with horseradish peroxidase (HRP; from Sino Biological) was added and incubated for 1 hour at room temperature. A final wash step was performed, followed by addition of 3,3′5,5′-tetramethylbenzidine substrate (TMB, from Thermo Fisher Scientific). The reaction was stopped after 25 minutes by TMB stop solution (Scytek Laboratories, Logan, UT, USA). The optical density (OD) of the chromogenic signal is directly proportional to the amount of anti-rS IgG2 captured on the plate, providing a quantifiable measurement of IgG3 concentration in the serum sample. Anti-Spike IgG3 concentration was calculated by interpolating to the levels of the IgG3 reference standard curve.

Total Anti-Spike IgG assay: Assay plates (Thermo Fisher Scientific, Waltham, MA, USA) were coated with 1-2 μg/mL SARS-CoV-2 rS protein (produced at Novavax, Inc., Gaithersburg, MD, USA) for 15 to 72 hours at 2-8° C. Plates were then washed with phosphate-buffered saline with Tween 20 (PBST) and blocked for 1 hour with blocking buffer (Thermo Fisher Scientific). Human serum samples (reference standards, quality controls, or test sera) were then added to the wells, allowing anti-rS protein IgG antibodies to bind to S protein coated on plate (2 hours of incubation). An anti-SARS-CoV-2 Spike RBD IgG monoclonal antibody (Acro Biosystems, Cat #RAS008-02) was used as a reference standard. The plates were again washed with PBST, and then a goat anti-human IgG secondary antibody conjugated with horseradish peroxidase (HRP; from Southern Biotech, Birmingham, AL, USA) was added and incubated for 1 hour at room temperature. A final wash step was performed, followed by addition of 3,3′5,5′-tetramethylbenzidine substrate (TMB, from Thermo Fisher Scientific). The reaction was stopped after 20 minutes by TMB stop solution (Scytek Laboratories, Logan, UT, USA). The optical density (OD) of the chromogenic signal is directly proportional to the amount of Total anti-rS IgG captured on the plate, providing a quantifiable measurement of Total IgG concentration in the serum sample. Total anti-Spike IgG concentration was calculated by interpolating to the levels of the IgG reference standard curve.

Calculation of % IgG4: Data for anti-Spike IgG4, IgG1 and total IgG for individual subjects were tabulated. Anti-Spike IgG4(%) were calculated by anti-Spike IgG4/Total anti-spike IgG*100 for each subject. Anti-Spike IgG1(%) were calculated by anti-Spike IgG1/Total anti-spike IgG*100 for each subject.

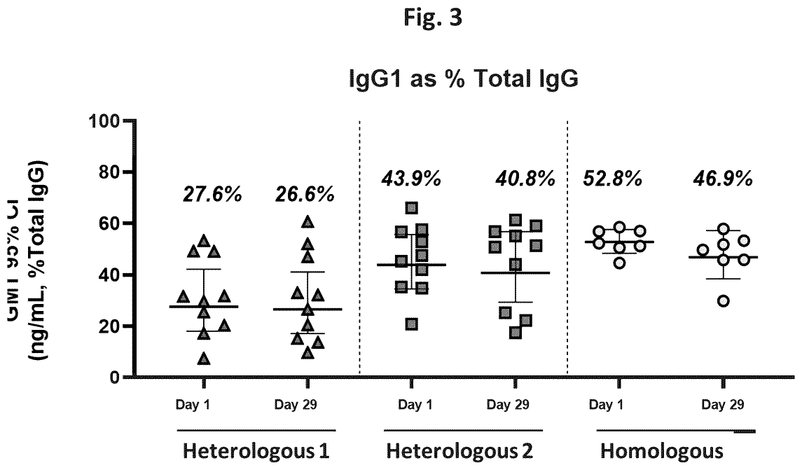

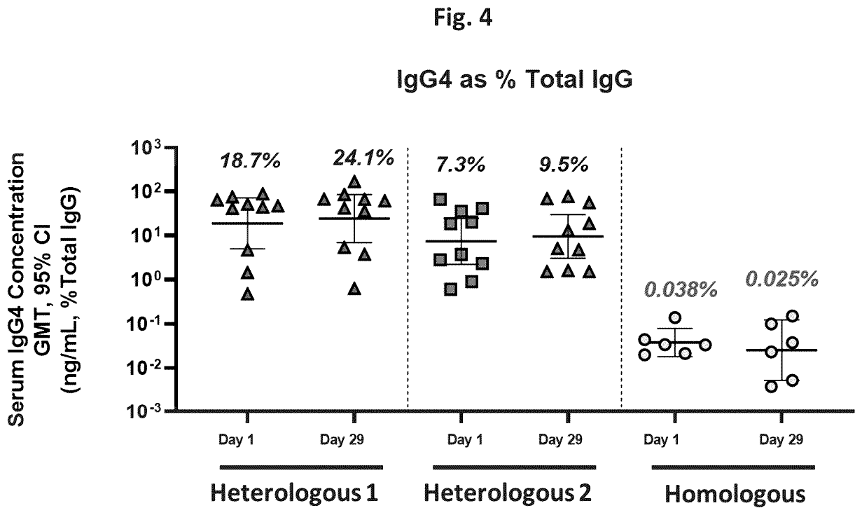

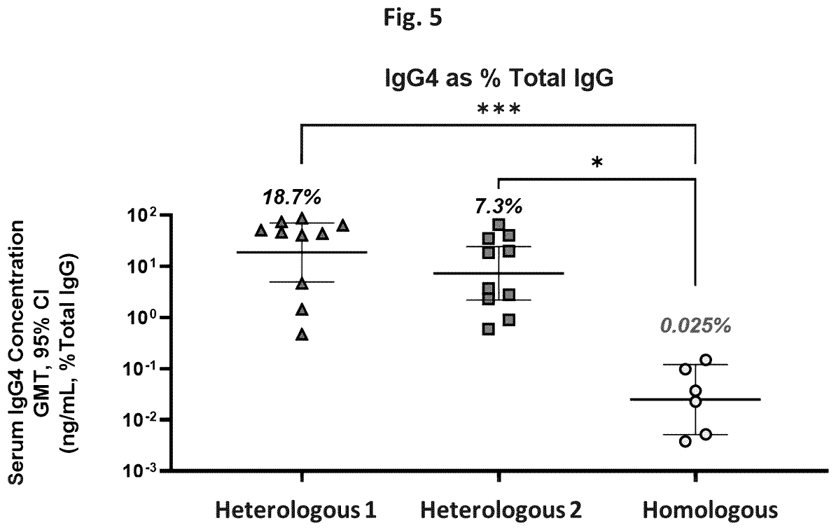

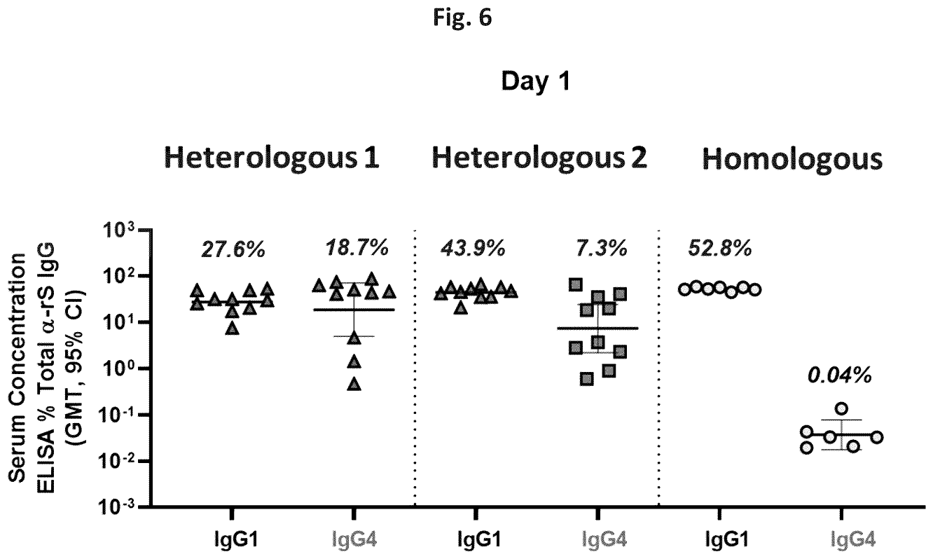

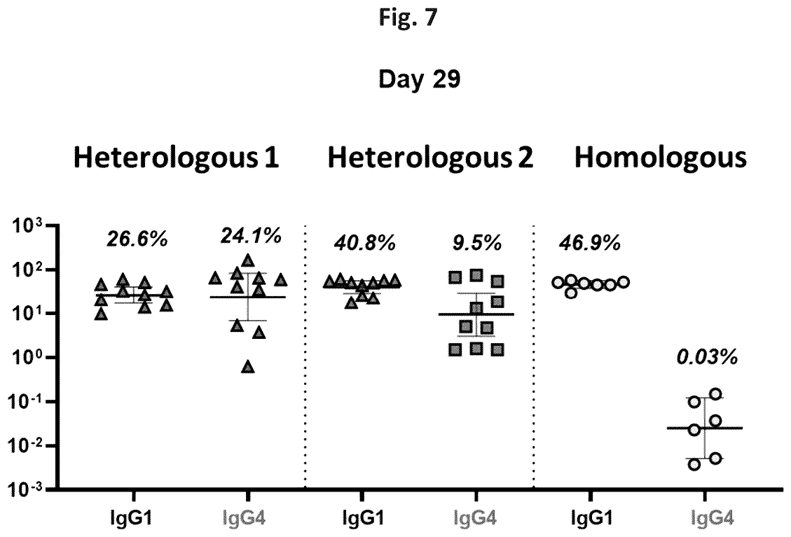

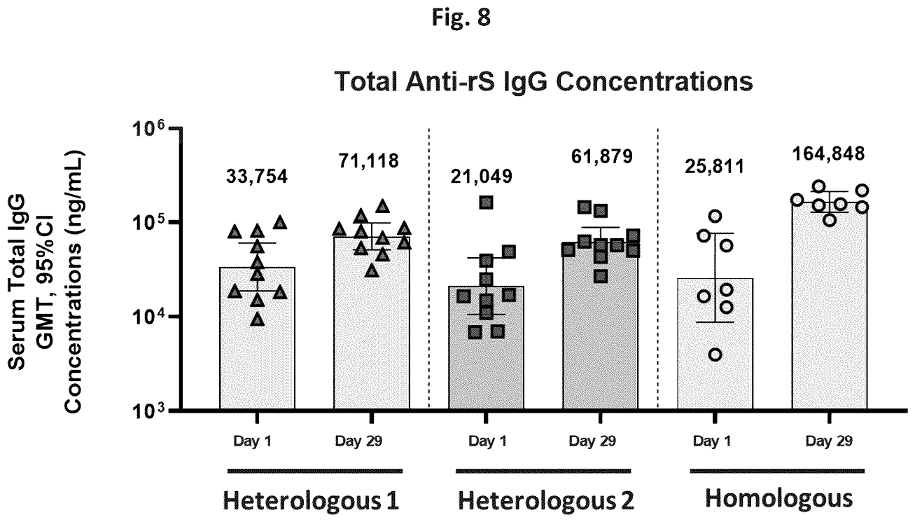

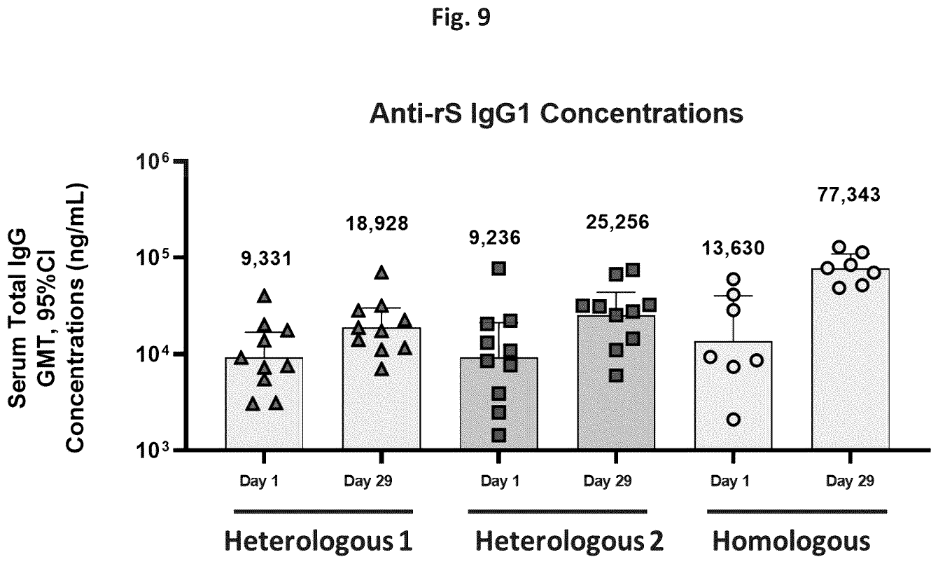

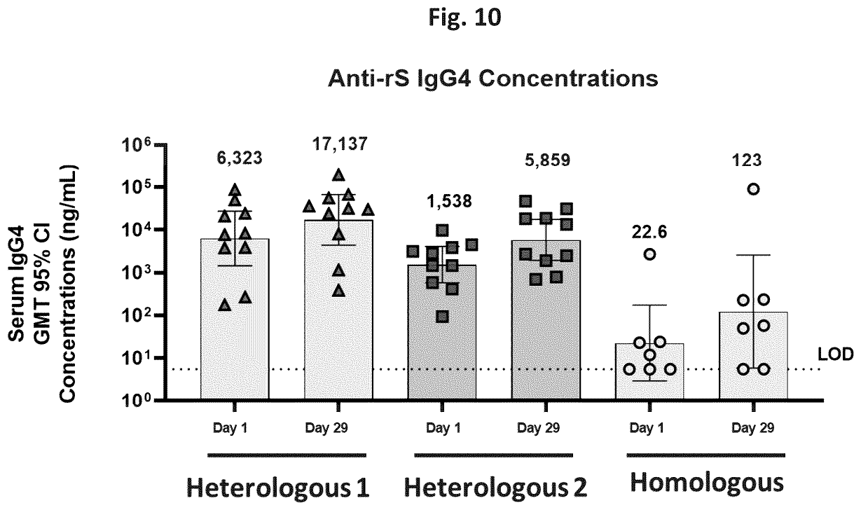

Results: FIG. 3 shows the % of IgG1 out total IgG in the samples at day 1 (before boost) and at day 29 (28 days after boost). FIG. 4 shows the % of IgG4 out total IgG in the samples at day 1 (before boost) and at day 29 (28 days after boost). FIG. 5 shows the % of IgG4 out total IgG in the samples at day 29 (28 days after boost). FIG. 6 shows the % of IgG1 and IgG4 in the samples at day 1 (before boost). FIG. 7 shows the % of IgG1 and IgG4 in the samples at day 29 (28 days after boost). FIG. 8 shows the total anti-S glycoprotein IgG in the samples. FIG. 9 shows the total anti-S glycoprotein IgG1 in the samples. FIG. 10 shows the total anti-S glycoprotein IgG4 in the samples.

Table A shows the % of IgG4 out of total IgG in each sample on day 1 (D1 in the table) and day 29 (D29 in the table). Table B shows the % of IgG1 out of total IgG in each sample on day 1 (D1 in the table) and day 29 (D29 in the table). Table C shows the total anti-S IgG concentration in each sample on day 1 (D1 in the table) and day 29 (D29 in the table). Table D shows the total anti-S IgG1 concentration in each sample on day 1 (D1 in the table) and day 29 (D29 in the table). Table E shows the total anti-S IgG concentration in each sample on day 1 (D1 in the table) and day 29 (D29 in the table).

TABLE A

| | Heterologous | Heterologous | Heterologous | Heterologous | Homologous | Homologous |
|---|---|---|---|---|---|---|
| | 1 D1 | 1 D29 | 2 D1 | 2 D29 | D1 | D29 |
| n | 10 | 10 | 10 | 10 | 6 | 6 |
| Geometric | 18.7 | 24.1 | 7.3 | 9.5 | 0.04 | 0.03 |
| mean | | | | | | |
| Lower | 5.0 | 6.8 | 2.2 | 3.0 | 0.02 | 0.01 |
| 95% CI | | | | | | |
| Upper | 70.4 | 84.6 | 24.3 | 29.4 | 0.08 | 0.12 |
| 95% CI | | | | | | |

TABLE B

| | Heterologous | Heterologous | Heterologous | Heterologous | Homologous | Homologous |
|---|---|---|---|---|---|---|
| | 1 D1 | 1 D29 | 2 D1 | 2 D29 | D1 | D29 |
| n | 10 | 10 | 10 | 10 | 7 | 7 |
| Geometric | 27.6 | 26.6 | 43.9 | 40.8 | 52.8 | 46.9 |
| mean | | | | | | |
| Lower | 18.1 | 17.2 | 34.6 | 29.3 | 48.4 | 38.4 |
| 95% CI | | | | | | |
| Upper | 42.2 | 41.1 | 55.7 | 56.8 | 57.6 | 57.2 |
| 95% CI | | | | | | |

TABLE C

| | Heterologous | Heterologous | Heterologous | Heterologous | Homologous | Homologous |
|---|---|---|---|---|---|---|
| | 1 D1 | 1 D29 | 2 D1 | 2 D29 | D1 | D29 |
| n | 10 | 10 | 10 | 10 | 7 | 7 |
| Geometric | 33,754 | 71,118 | 21,049 | 61,879 | 25,811 | 164,848 |
| mean | | | | | | |
| Lower | 18,832 | 51,221 | 10,539 | 43,299 | 8,726 | 127,979 |
| 95% CI | | | | | | |
| Upper | 60,500 | 98,744 | 42,040 | 88,432 | 76,346 | 212,340 |
| 95% CI | | | | | | |

TABLE D

| | Heterologous | Heterologous | Heterologous | Heterologous | Homologous | Homologous |
|---|---|---|---|---|---|---|
| | 1 D1 | 1 D29 | 2 D1 | 2 D29 | D1 | D29 |
| n | 10 | 10 | 10 | 10 | 7 | 7 |
| Geometric | 9,331 | 18,928 | 9,236 | 25,256 | 13,630 | 77,343 |
| mean | | | | | | |
| Lower | 5,157 | 11,910 | 4,033 | 14,530 | 4,619 | 55,184 |
| 95% CI | | | | | | |
| Upper | 16,883 | 30,082 | 21,155 | 43,898 | 40,219 | 108,400 |
| 95% CI | | | | | | |

TABLE E

| | Heterologous | Heterologous | Heterologous | Heterologous | Homologous | Homologous |
|---|---|---|---|---|---|---|
| | 1 D1 | 1 D29 | 2 D1 | 2 D29 | D1 | D29 |
| n | 10 | 10 | 10 | 10 | 7 | 7 |
| Geometric | 9,331 | 18,928 | 9,236 | 25,256 | 13,630 | 77,343 |
| mean | | | | | | |
| Lower | 5,157 | 11,910 | 4,033 | 14,530 | 4,619 | 55,184 |
| 95% CI | | | | | | |
| Upper | 16,883 | 30,082 | 21,155 | 43,898 | 40,219 | 108,400 |
| 95% CI | | | | | | |

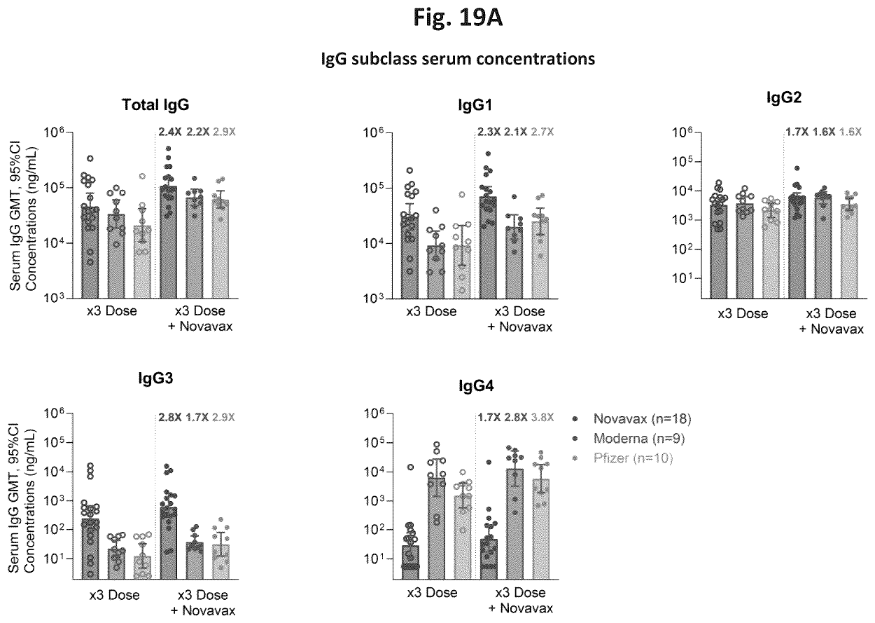

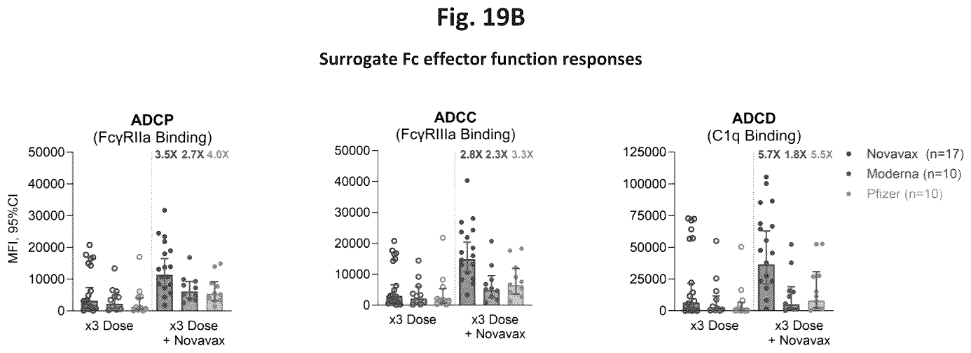

Total anti-S IgG and IgG1 levels following three homologous doses of mRNA or NVX-CoV2373 were similar, although NVX-CoV2373 induced somewhat higher levels, and the fourth dose of NVX-CoV2373 led to increased responses in each group (FIG. 19A). Compared with recipients of prior mRNA vaccine, anti-S IgG3 levels were markedly higher (>10-fold) after three or four homologous doses of NVX-CoV2373. By contrast, much higher anti-S IgG4 levels (>75-fold) were observed following repeated mRNA vaccination, but not after three or four homologous doses of NVX-CoV2373 (FIG. 19A). The fourth dose of NVX-CoV2373 also appeared to enhance surrogate signals for ADCP, ADCC, and ADCD activities in recipients of prior mRNA vaccine, though the effect was greater after a fourth homologous dose of NVX-CoV2373 (FIG. 19B).

The data that the NVX-CoV2373 rS protein vaccine does not appear to induce notable increases in IgG4, even after multiple exposures, or to impair Fc-dependent effector responses as observed with mRNA vaccines. Instead, NVX-CoV2373 drove proportional increases in IgG3, perhaps the most potent SARS-CoV-2 neutralizing antibody subclass, and enhanced surrogate ADCP, ADCD, and ADCC activity.

The data shows the ability of the assays to detect IgG4 class switch in samples of patients that received COVID-19 vaccines. Specifically, the data shows that there is significantly lower IgG4 in serum samples from patients that received vaccines containing SARS-CoV-2 S glycoproteins than in serum samples from patients that received vaccines containing mRNA encoding a SARS-CoV-2 S glycoprotein.

### Example 2: Assays to Measure Anti-rS Proteins IgG in Serum Against SARS-CoV-2 Ancestral and Variant Strain rS Proteins

Purpose: The anti-rS proteins I IgG assays were developed to quantify vaccine response and establish correlation of such responses with protection against SARS-CoV-2 variant strains. The assay was assessed for precision, specificity, linearity, and other validation parameters, as well as correlation with pseudovirus neutralization, wild-type virus neutralization, and hACE2 binding inhibition assay results.

Samples: Healthy human serum samples from before the COVID-19 pandemic (collected in 2016 through 2018) were obtained from BioIVT (Westbury, NY, USA) and Valley Biomedical (Winchester, VA, USA). COVID-19 convalescent serum was obtained from Sanguine BioSciences (Waltham, MA, USA) and BioIVT. Serum samples obtained from the Novavax clinical trial repository were from participants in phase 1 to 3 trials of the COVID-19 vaccine NVX-CoV2373 (Novavax, Gaithersburg, MD, USA). Positive quality control (QC) samples (COVID-19 convalescent serum pools) known to have high or low anti-rS IgG levels were used. Negative controls (NC) were pre-COVID-19 sera negative for anti-rS IgG. QC samples were tested in duplicate wells on the first plate of each run. For correlation analyses, serum samples were from the Novavax clinical trial 2019nCoV-311 (NCT05372588). For analysis of conversion to WHO international units, the following samples were used: high QC (HQC)/low QC (LQC)/NC samples (as described above), an in-house reference standard COVID-19 convalescent serum pool, WHO international standard (NIBSC code 20/136) [10,11], and the WHO reference panel (NIBSC code 20/268). The reference panel contains 5 different pooled samples, ranging from high to low and negative antibody titers.

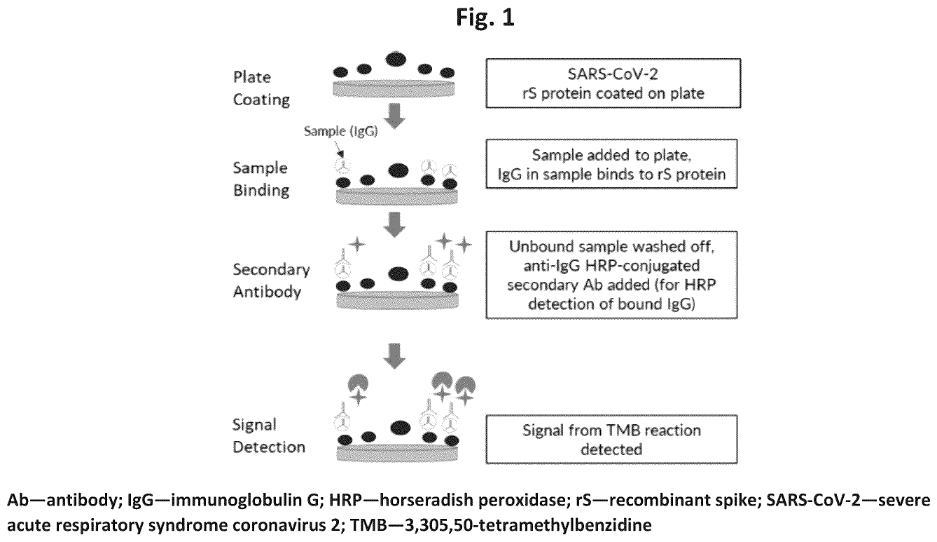

Assay Procedure: Each sample was subjected to an anti-Spike IgG4 assay (FIG. 1). Assay plates (Thermo Fisher Scientific, Waltham, MA, USA) were coated with 0.80 μg/mL SARS-CoV-2 rS protein (produced at Novavax, Inc., Gaithersburg, MD, USA) for 15 to 72 h at 2-8° C. For assay development and initial validation, Novavax's SARS-CoV-2 rS protein was prepared from the full-length (1273 amino acid), wild-type ancestral SARS-CoV-2 S protein based on Genbank gene sequence MN908947, nucleotides 21563-25384. A stable pre-fusion protein was produced by mutating the furin cleavage site and two residues in the CH domain. The recombinant protein was expressed in Sf9 insect cells and chromatographically purified to yield homotrimers displaying the N-terminal domain and receptor binding domain on the apical surface, as previously shown [9]. Coated plates were then washed with phosphate-buffered saline with Tween 20 (PBST) and blocked for 1 h with a blocking buffer (Thermo Fisher Scientific, Cat #37542). Human serum samples (reference standards, quality controls, or test sera) were then added to the wells, allowing anti-rS protein IgG antibodies to bind (2 h of incubation). The plates were again washed with PBST, and then a goat anti-human IgG secondary antibody conjugated with horseradish peroxidase (HRP) (SouthernBiotech, Cat #2040-05) was added and incubated for 1 h at room temperature. A final wash step was performed, followed by the addition of 3,305,50-tetramethylbenzidine substrate (TMB, Sigma, Cat #T00440-1L). The reaction was stopped after 10 min by TMB stop solution (Scytek Laboratories, Cat #TSB999). The optical density (OD) of the chromogenic signal is directly proportional to the amount of anti-rS IgG captured on the plate, providing a quantifiable measurement of rS-specific IgG concentration in the serum sample.

Validation Assays (Precision): Twenty-seven samples were tested twice per assay run (in duplicate) in 6 different assay runs performed by 3 different analysts on 2 different days. The geometric mean concentration (GMC) was calculated for each sample from the 6 runs. Precision was then estimated by calculating the percent geometric coefficient of variation (% GCV), based on the variance component analysis using analyst and day as random effects and the samples as a fixed effect. The acceptance criteria for precision were that at least 80% of samples should have a % GCV<20%, while samples with IgG concentrations at or near the lower limit of quantitation (LLoQ) were permitted to have a % GCV<25%.

Validation Assays (Selectivity): Forty samples collected before the COVID-19 pandemic (which are thus expected to be negative for SARS-CoV-2-specific antibodies) were tested in the IgG assay. These samples were expected to have concentrations below LLoQ.

Validation Assays (Specificity): To confirm the specific detection of anti-rS IgG, homologous antigen competition was assessed by incubating anti-rS IgG-positive samples with different amounts of SARS-CoV-2 rS for 1 h at room temperature before testing. Controls were samples incubated with assay buffer only. The acceptance criterion was that homologous protein incubation should reduce the detected IgG concentration by at least 50% in at least 80% of the samples tested. To assess the potential for cross-reactivity with other betacoronavirus S proteins, samples were incubated with S protein from SARS-CoV-1 and MERS-CoV. Samples were also incubated with irrelevant proteins-respiratory syncytial virus fusion protein (RSV F) at 4_g/mL and influenza virus hemagglutinin (HA) (A/Kansas/14/2017 virus like particles [VLP]) at 4 HA units. These related and irrelevant viral proteins were, like SARS-CoV-2 rS, expressed in Sf9 insect cells and purified by generally parallel chromatographic methods. The targeted result was that incubation with irrelevant protein should not reduce the detected IgG concentration (_20% reduction) in at least 80% of the samples tested. Additionally, pre-COVID-19 samples from 5 participants vaccinated against RSV (Novavax clinical trial RSV-M-301, NCT02624947) and 5 participants vaccinated against influenza (Novavax clinical trial qNIV-E-301, NCT04120194) were tested in the anti-rS IgG assay (both pre- and post-vaccination samples). The targeted results were that vaccination against RSV or influenza using antigens produced in the same vaccine platform would not cause changes in detectable anti-rS IgG and that levels of anti-rS IgG should be low in the post-immunization samples despite vigorous responses to other highly immunogenic respiratory viral proteins. For all specificity outcomes, the percent reduction in IgG concentration (% inhibition) was calculated as follows:

$$\% Inhibition = [ 100 - \frac{Results with protein incubation}{Results without protein incubation} \times 100 ]$$

Validation Assays (Matrix Effects): To assess the impact of hemolysis on the assay, 100% hemolyzed human blood (BioIVT BRH1369895) was spiked into 6 samples, and negative control to produce 50% hemolyzed or 25% hemolyzed samples (to represent severe hemolysis). After being tested in the IgG assay, percent recovery was calculated (result for the hemolyzed sample divided by the result from the non-hemolyzed sample). The acceptance criteria were a percent recovery between 80 and 120% of the reference value and that the NC should remain <LLoQ. The same process was used to assess the impact of lipemia, with high lipemic serum with a high level of triglycerides (BioIVT BRH1119533, triglycerides 1473 mg/dL) being spiked into samples. The final triglyceride concentrations were 500 or 250 mg/dL (normal level, <150 mg/dL). The samples were then used in the IgG assay, and percent recovery was calculated. The acceptable range was recovery between 80 and 120% of the reference value, with the NCs below LLoQ.

Validation Assays (Linearity): Two phase 1 trial samples with high anti-rS IgG levels were tested in the assay undiluted and in a 1:2 dilution series (6 assay runs). Precision was calculated for each dilution point, and linear regression was performed for observed versus expected GMC. The expected EU/mL at each dilution was calculated from the overall GMC from all runs of the least diluted divided by the dilution factor for each dilution of each sample. The observed anti-rS protein IgG EU/mL at each dilution is the overall GMC at each dilution for each sample in all runs. To assess the ability of the assay to return values that accurately reflect the neat sample, the % relative bias at each dilution point was calculated as follows:

$$\% Relative Bias = 100 \times \frac{Observed overall anti rS IgG GMC - Expected anti rS IgG GMC}{Expected anti - rS IgG GMC}$$

Validation Assays (Sensitivity): The lowest IgG level values that were accurately and precisely determined (as above) were assessed for the 2 linearity assessment samples in the linearity analysis. The LLoQ for the assay was set at 200 EU/mL during assay qualification and was confirmed during validation.

Validation Assays (Assay Robustness (Incubation Time and Plate Coating Time)): Incubation time robustness—The assay was conducted on 18 samples using lower and upper time limits for each incubation step as follows: plate coating time (15 and 72 h, lower and upper time limit, respectively), plate blocking time (60 min and 90 min), sample incubation on plate (110 min and 130 min), secondary antibody HRP on the plate (50 min and 70 min), TMB incubation on plate (8 min and 12 min). Assay results were then compared with the precision analysis runs performed under reference conditions. The acceptance criteria were that >80% of samples should have values within 80-120% of reference values. Percent recovery was calculated as follows:

$$\% Recovery = 100 \times \frac{Test condition value}{Reference condition value}$$

Validation Assays (Sample Stability): Samples were stored at various temperatures: 6, 24, or 48 h at room temperature, 6 or 7 days at 2-8° C., 29 days at −20±10° C., 6 or 24 months at −80±10° C. Samples were then tested in the assay, and the results were compared with the reference condition results from the precision analysis assay runs. Samples were also tested after 3, 6, 7, or 8 freeze/thaw cycles (1 h at room temperature followed by refreezing), and results were compared to the precision run results (only 1 freeze/thaw cycle). Percent recovery was calculated as shown above, with acceptable recovery between 80-120% of reference.

Validation Assays: The assay was also adapted for Beta, Delta, Omicron BA.1, Omicron BA.5, and Omicron XBB.1.5 variants. The assay method described in FIG. 1 is the same except the ancestral S protein used to coat the plate was replaced by the relevant variant sequence protein. A similar validation process as described in FIG. 1 was followed.

Correlation Analyses: Each sample was assessed in the anti-rS IgG assay described here. The same samples were also tested in wild-type virus neutralization, pseudovirus neutralization, and hACE2 binding inhibition assays.

Results: The IgG assay is precise, robust, linear, and specific for SARS-CoV-2 S protein and is useful for both ancestral strain and variants (tested for Beta, Delta, and Omicron BA.1/BA.5/XBB.1.5). Results from the IgG assay correlated significantly with pseudovirus/live wild-type virus neutralization and hACE2 binding inhibition assays for all variants tested, ranging from the ancestral virus to XBB.1.5.

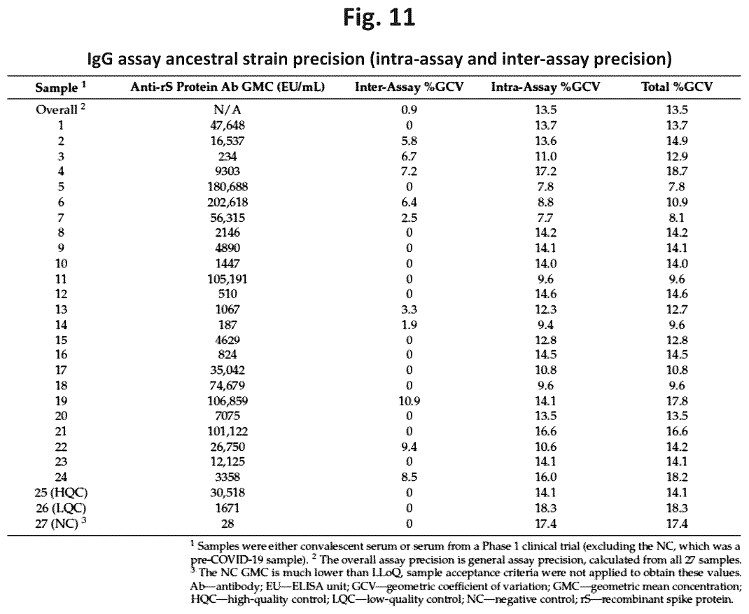

Inter-assay, intra-assay, and total precision were <20% GCV for all 27 serum samples representing all concentration ranges (low, medium, and high concentrations) are shown in FIG. 11. Assay plates (Thermo Fisher Scientific, Waltham, MA, USA) were coated with 0.80 μg/mL SARS-CoV-2 rS protein (produced at Novavax, Inc., Gaithersburg, MD, USA) for 15 to 72 h at 2-8° C.

Assay selectivity met acceptance criteria of 80% of samples having IgG levels below LLoQ. Of 40 serum samples collected before the COVID-19 pandemic (collected in 2016 through 2018), 35 had anti-rS IgG levels below LLoQ of 200 EU/mL (Table S1). Some of these samples were known to have high influenza hemagglutination inhibition assay titers (data not shown). Thus, the IgG assay is not affected by the presence of antibodies to other common respiratory pathogens.

Assay specificity also met the acceptance criteria. When 6 samples (from a phase 1 trial) were incubated with SARS-CoV-2 rS protein, IgG detection was strongly inhibited by >50% for all 6 samples, indicating that the assay is specific to SARS-CoV-2 rS protein (Table 2). When the same 6 samples were incubated with rS proteins of MERS-CoV and SARS-CoV-1, the phase 1 trial sera showed much less inhibition of the IgG signal, suggesting little crossreactivity with MERS-CoV and SARS-CoV-1 in the assay. We did not test cross-reactivity with HCoV-OC34 or HKU1 sequence spike proteins, but these proteins have significantly lower sequence homology with SARS-CoV-2 spike than SARS-CoV-1 and MERS-CoV and have been shown to produce only minimal levels of cross-reactivity (16). Here, the absence of signals significantly >LLoQ in pre-pandemic sera, despite the widespread seropositivity to the endemic seasonal coronaviruses, is consistent with minimal cross-reaction.

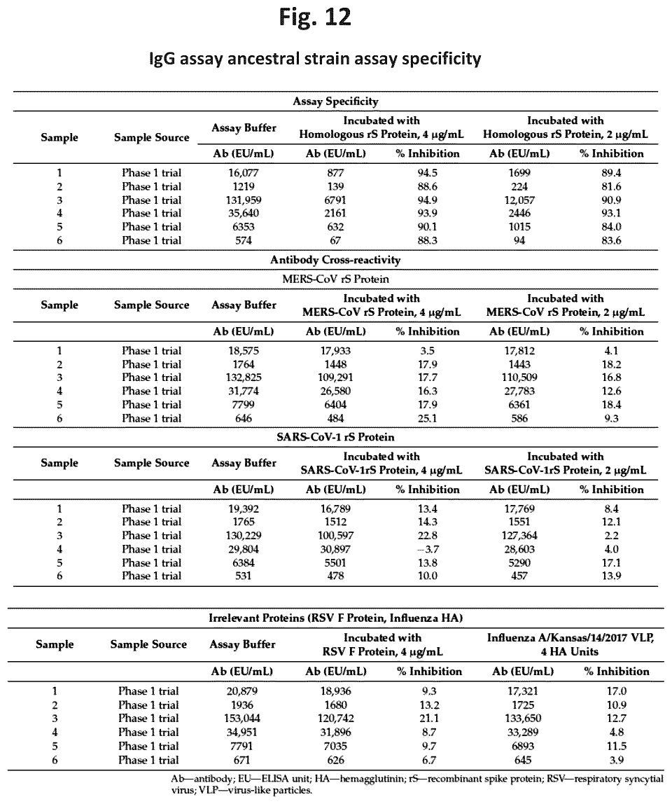

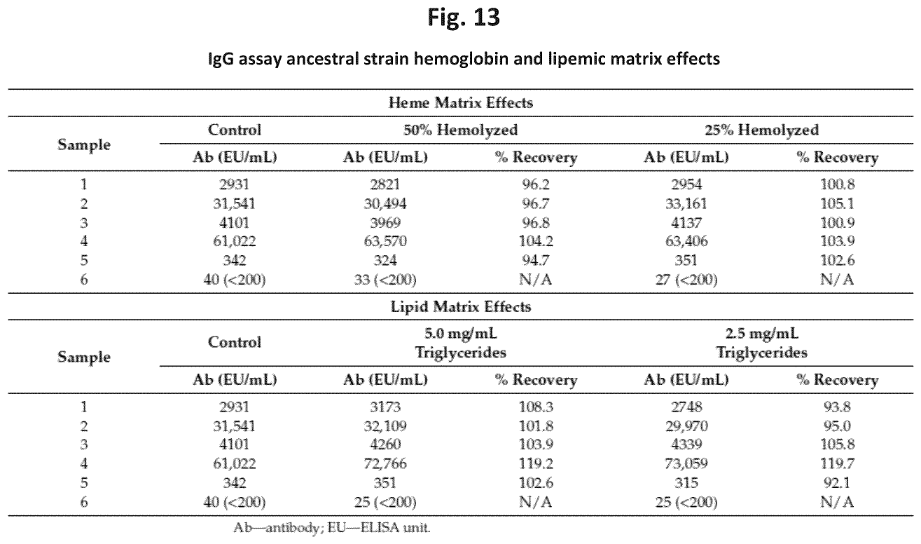

The same 6 samples were also incubated with irrelevant proteins produced on the same vaccine platform (RSV F protein or influenza virus-like particles [VLP]) are shown in FIG. 12. Among clinical trial samples, there was no significant inhibition (defined as >20%) of IgG detection by irrelevant proteins for 5 of 6 (83.6%) samples for RSV incubation and for 6 of 6 (100%) samples for influenza VLP. Additionally, among 5 participants vaccinated against RSV and 5 vaccinated against influenza, anti-rS IgG levels (using immunogens manufactured on the same insect cell platform) did not change in post-vaccination versus pre-vaccination samples (Table S2). One RSV-vaccinated individual showed a 2.26-fold increase in anti-rS IgG concurrent with immunization, but the increase was from a <LLoQ value to a value slightly above LLoQ. Free hemoglobin and lipemic matrix had minimal impact on the assay are shown in FIG. 13.

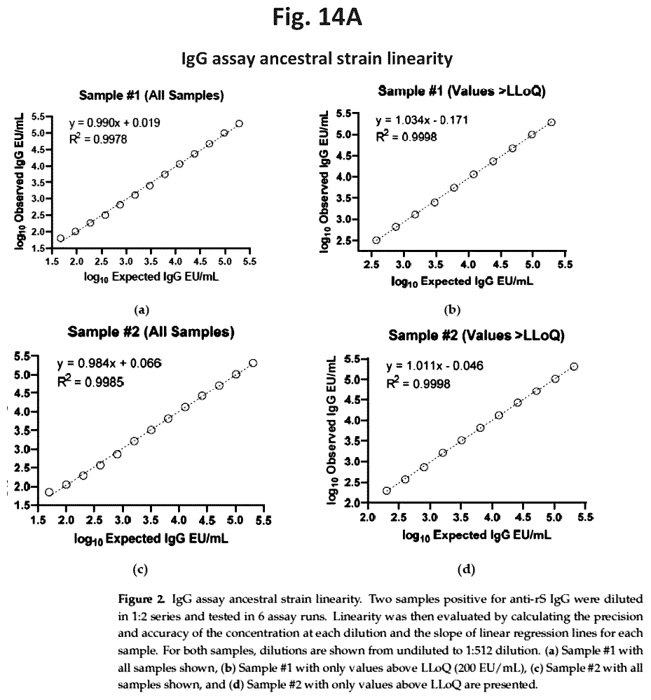

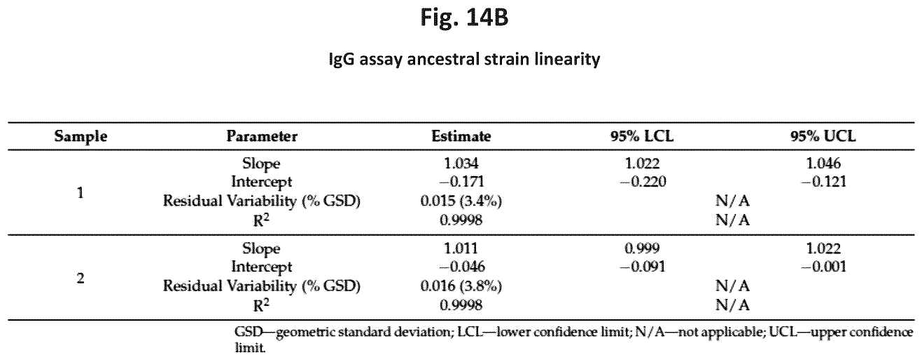

Linearity of the assay was demonstrated, with R2 values of 0.9998 for the 2 tested samples are shown in FIG. 14A and FIG. 14B. LLoQ was assigned at 200 EU/mL, based on the lowest concentration values that could be detected accurately and precisely in these two samples (and precision data [FIG. 11] on samples with anti-rS levels approximating 200 EU/mL supported this selection). Based on phase 1 samples, the upper limit of quantitation (ULoQ) was provisionally assigned as at least 206,767 EU/mL. However, based on 5 serum samples from phase 2 and phase 3 trials of the COVID vaccine NVX-CoV2373, the ULoQ is currently estimated to be at least 2,904,275 EU/mL.

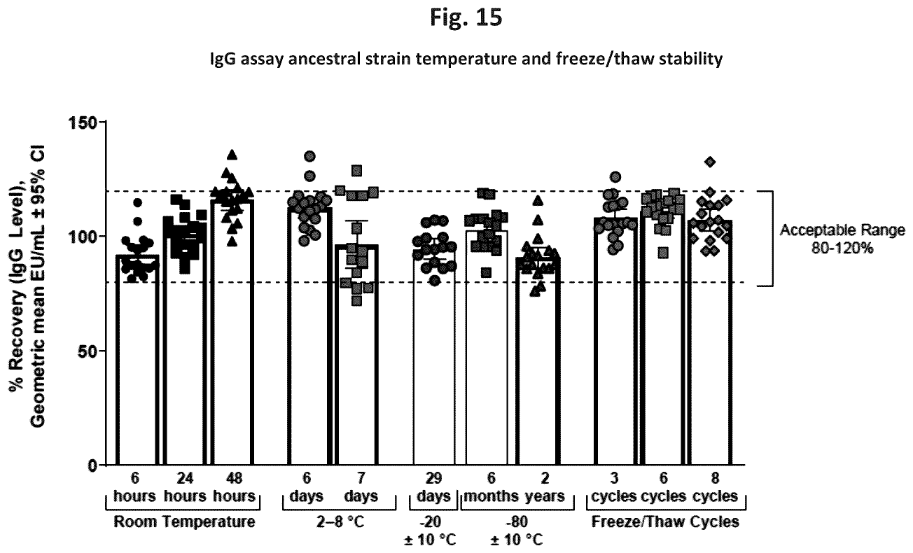

The stability of samples was tested at different storage temperatures (room temperature, refrigeration [2-8° C.], and freezing [−20° C. or −80° C.]). Samples were also assessed after multiple freeze/thaw cycles are shown in FIG. 15. The samples were stable up to 8 freeze/thaw cycles and up to 2 years in −80° C. freezer storage, as shown by recovery within the acceptable recovery range of 80-120% of reference.

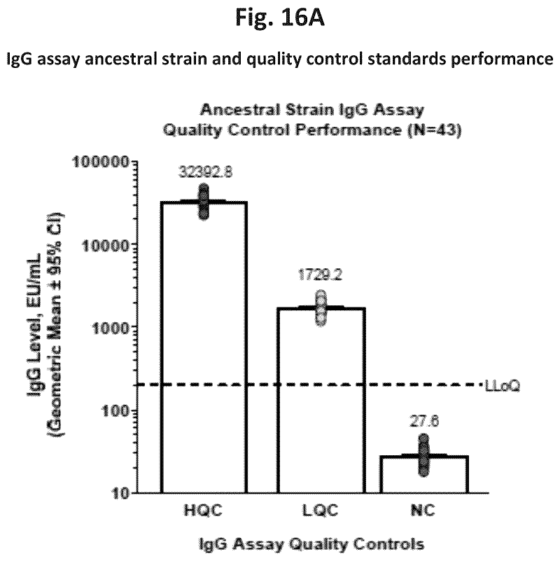

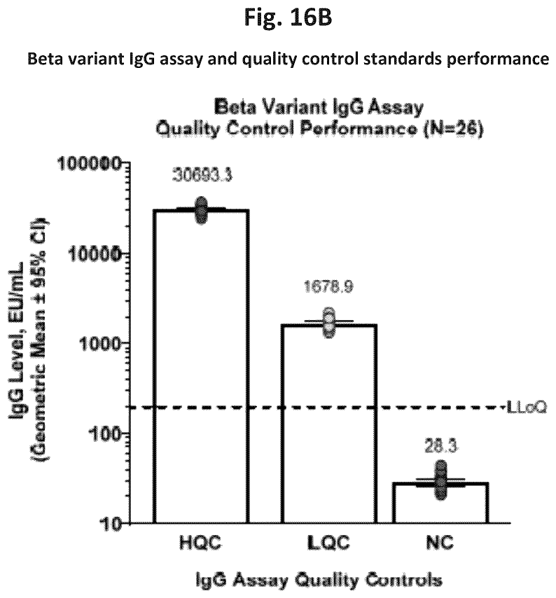

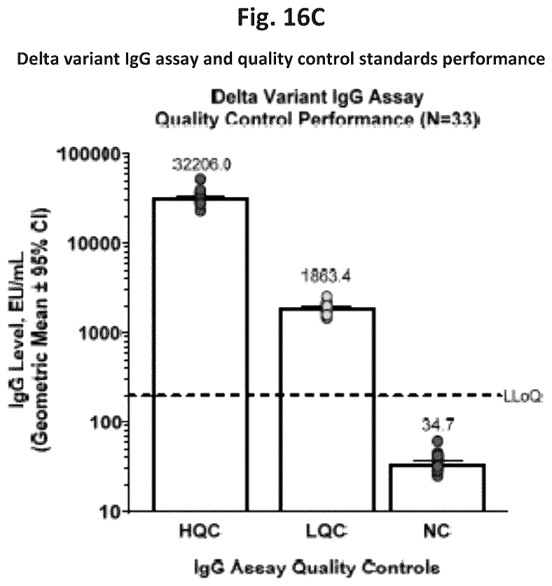

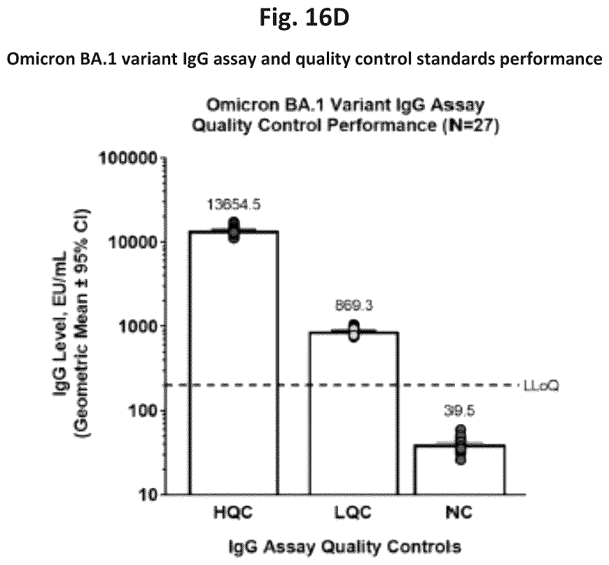

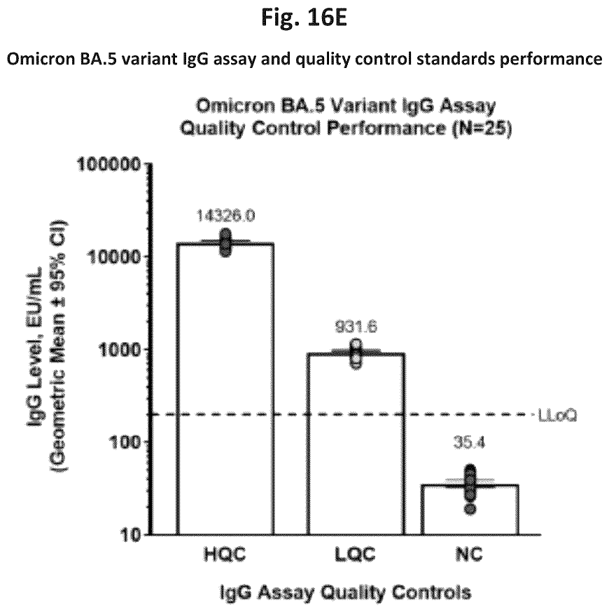

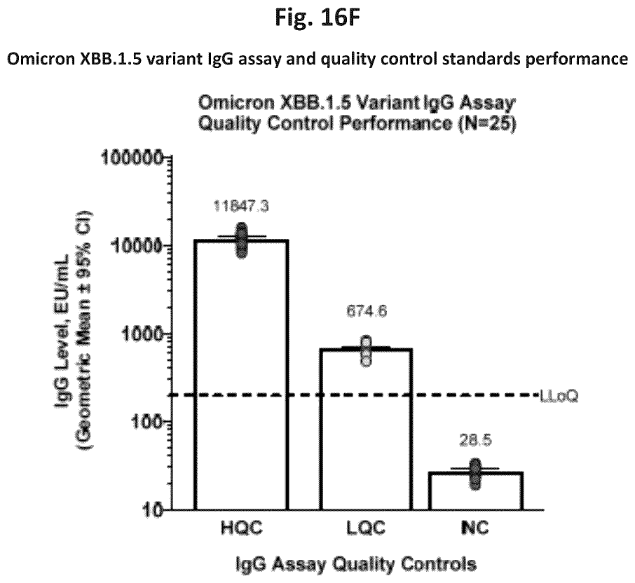

Results for the assay validation parameters for the Beta, Delta, and Omicron BA.1/BA.5/XBB.1.5 variants were similar to those for the ancestral strain. Quality control samples performed similarly well for variants compared to ancestral strain as shown in FIG. 16A. For the Beta variant, inter-assay, intra-assay, and total precision had <20% GCV for 18 of 20 (90%) samples. Selectivity was demonstrated, as 36 of 40 (90.0%) pre-COVID-19 samples showed <LLoQ results in the assay (FIG. 16B). Specificity was also demonstrated, with 8 of 8 samples showing >50% reduction in IgG detection when incubated with homologous S protein. SARS-CoV-1 and MERS-CoV S protein reduced IgG detection in 5 of 8 and in 3 of 8 samples, respectively. RSV F protein reduced IgG detection in 3 of 8 samples; influenza hemagglutinin did not reduce IgG detection in any samples. Among 5 participants vaccinated against RSV and 5 vaccinated against influenza, none of the post-vaccination samples showed changes in anti-rS IgG detection compared with pre-vaccination. Linearity was successfully demonstrated (R2=0.9999 and 0.9996), with LLoQ defined as 200 EU/mL and ULoQ determined to be at least 490,731 EU/mL. For the Delta variant, 19 of 20 (95%) samples had inter-assay, intra-assay, and total precision % GCV <20%. Of 40 pre-COVID-19 serum samples, 33 (82.5%) showed <LLoQ results in the assay. Of 7 samples, 6, 3, and 7 samples showed >50% reduction in IgG detection when incubated with homologous S proteins (ancestral, Beta, and Delta, respectively) (FIG. 16C). SARS-CoV-1 and MERS-CoV S protein reduced IgG detection in 2 of 7 and in 1 of 8 samples, respectively. RSV F protein did not reduce IgG detection in any samples; influenza hemagglutinin reduced IgG detection in 1 of 7 samples. Among 5 participants vaccinated against influenza, none of the post-vaccination samples had changes in anti-rS IgG compared with pre-vaccination. Of the 5 participants vaccinated against RSV, only 1 showed changes in anti-rS IgG levels. Linearity was successfully demonstrated (R2=0.9999 and 0.9998), LLoQ was defined as 200 EU/mL, and ULoQ was determined to be at least 501,789 EU/mL. For Omicron BA.1, inter-assay, intra-assay, and total precision had <20% GCV for 20 of 21 (95.2%) samples. Of 40 pre-COVID-19 serum samples, 31 (77.5%) showed <LLoQ results. Of 8 samples, 7 to 8 samples showed >50% reduction in IgG detection when incubated with homologous S protein. RSV F protein did not reduce IgG detection in 6 of 8 (75%) samples (FIG. 16D). Influenza hemagglutinin did not reduce IgG detection in 7 of 8 (87.5%) samples. Among 5 participants vaccinated against influenza, none of the post-vaccination samples had changes in anti-rS IgG compared with pre-vaccination. Of the 5 participants vaccinated against RSV, only 1 showed changes in anti-rS IgG levels. Linearity was successfully demonstrated (R2=0.986 and 0.966), LLoQ was defined as 200 EU/mL, and ULoQ was determined to be at least 391,124 EU/mL. Robustness for plate coating time was demonstrated, as 17 of 21 samples (81.0%) had recovery within 80-120% of the baseline values. For Omicron BA.5, all 21 samples had inter-assay, intra-assay, and total precision % GCV <20%. Of 40 pre-COVID-19 serum samples, 34 (85%) showed <LLoQ results. Of 8 samples, 6 to 8 samples showed >50% reduction in IgG detection when incubated with SARS-CoV-2 S protein (7/8 for Omicron BA.5, 6-7/8 for Omicron BA.1, 6/8 for ancestral strain) (FIG. 16E). RSV F protein did not reduce IgG detection in 7 of 8 (87.5%) samples; Ebola glycoprotein did not reduce IgG detection in any samples. Linearity was successfully demonstrated (R2=0.9988 and 0.9991). LLoQ was defined as 200 EU/mL, and ULoQ was determined to be at least 990,591 EU/mL. Robustness for plate coating time was demonstrated, as 17 of the 21 samples (81.0%) were within 80-120% of the baseline values. For Omicron XBB.1.5, all 21 serum samples had inter-assay, intra-assay, and total precision % GCV <20%. Of 40 pre-COVID-19 samples, 33 (82.5%) showed <LLoQ results. Of 8 samples tested, all samples showed a >50% reduction in IgG detection when incubated with SARS-CoV-2 S protein (ancestral strain, Omicron XBB.1.5, or Omicron BA.5) (FIG. 16F). Incubation with RSV F protein did not reduce IgG detection in 7 of 8 (87.5%) samples, and Ebola glycoprotein incubation did not reduce IgG detection in any of the 8 samples. Linearity was successfully demonstrated (R2=0.9985 and 0.9991). LLoQ was defined as 200 EU/mL, and ULoQ was determined to be at least 682,680 EU/mL. Robustness for plate coating time was demonstrated, as all 21 tested samples had recovery within 80-120% of the baseline values.

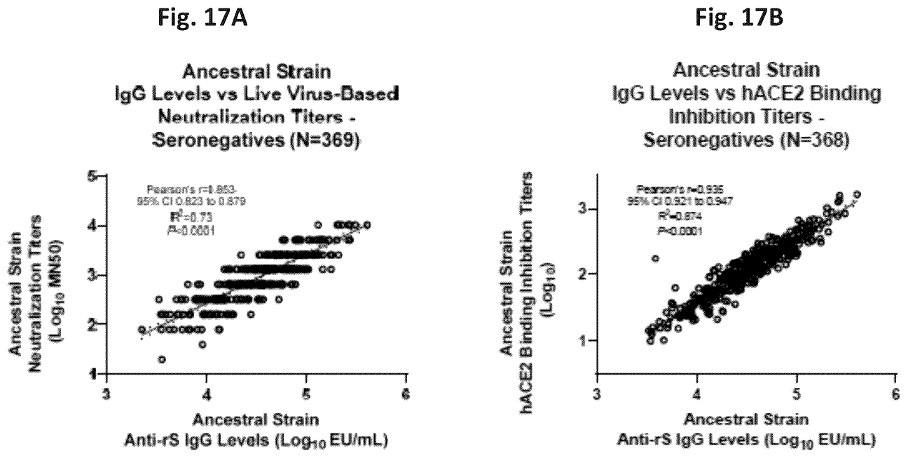

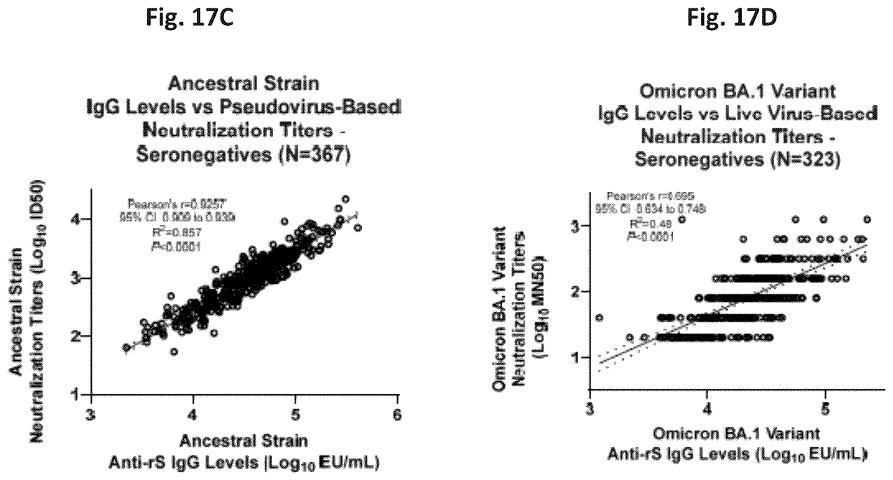

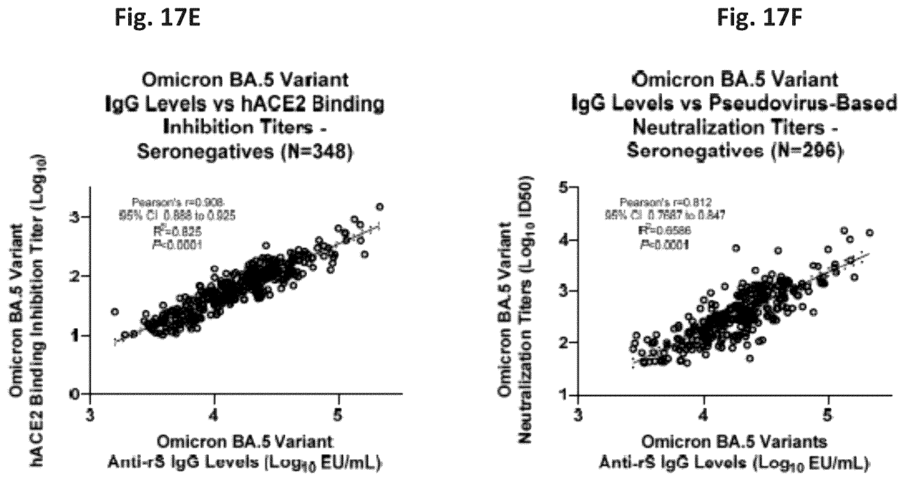

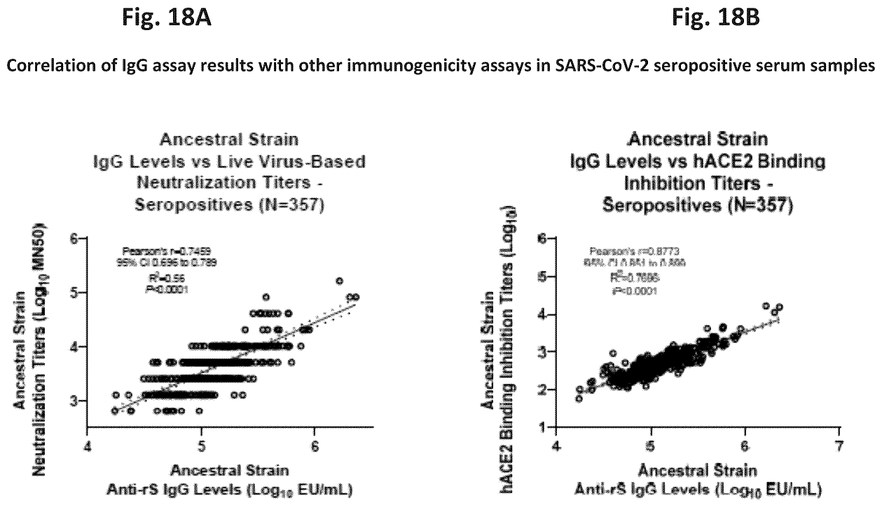

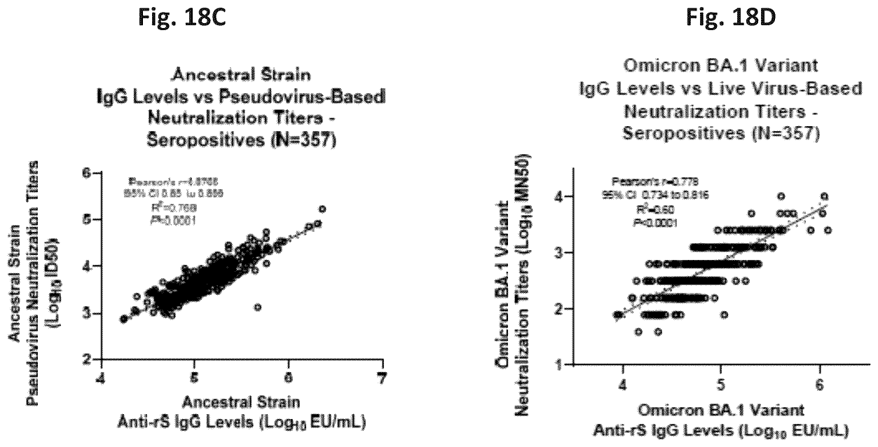

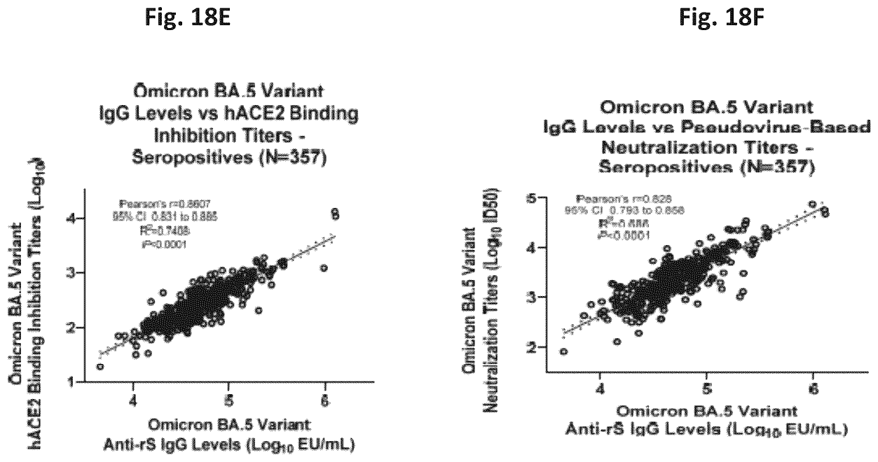

Natural SARS-CoV-2 infection also induces anti-rS IgG as part of the body's immune response. Levels of anti-rS IgG in the serum from both seropositive and seronegative subjects were evaluated using the anti-rS IgG assays for ancestral, Omicron BA.1, and Omicron BA.5 strains, followed by correlation with the neutralizing antibody levels (either live virus-based microneutralization assays or pseudovirus-based neutralization assays) and the hACE2 binding inhibition assay. For seronegative samples, the IgG assay ancestral strain results correlated significantly with results from the live wild-type virus neutralization assay (R2=0.73, Pearson's r=0.853, p<0.0001) (FIG. 17A), hACE2 binding inhibition assay (R2=0.874, Pearson's r=0.935, p<0.0001) (FIG. 17B), and pseudovirus-based neutralization assay (R2=0.857, Pearson's r=0.926, p<0.0001) (FIG. 17C). IgG assay Omicron BA.1 results correlated significantly with results from the live wild-type virus neutralization assay (R2=0.48, Pearson's r=0.695, p<0.0001) (FIG. 17D). IgG assay Omicron BA5 results correlated significantly with results from the hACE2 binding inhibition assay (R2=0.825, Pearson's r=0.906, p<0.0001) (FIG. 17E) and pseudovirus-based neutralization assay (R2=0.659, Pearson's r=0.812, p<0.0001) (FIG. 17F). Similarly, for seropositive samples, the IgG assay ancestral strain results correlated significantly with results from the live wild-type virus neutralization assay (R2=0.56, Pearson's r=0.746, p<0.0001) (FIG. 18A), hACE2 binding inhibition assay (R2=0.769, Pearson's r=0.877, p<0.0001) (FIG. 18B), and pseudovirus-based neutralization assay (R2=0.768, Pearson's r=0.876, p<0.0001) (FIG. 18C). IgG assay Omicron BA.1 results correlated significantly with results from the live wild-type virus neutralization assay (R2=0.60, Pearson's r=0.778, p<0.0001) (FIG. 18D). IgG assay Omicron BA5 results correlated significantly with results from the hACE2 binding inhibition assay (R2=0.741, Pearson's r=0.861, p<0.0001) (FIG. 18E) and pseudovirus-based neutralization assay (R2=0.686, Pearson's r=0.828, p<0.0001) (FIG. 18F).

Various modifications, equivalent processes, as well as numerous methods to which the present invention may be applicable will be readily apparent to those of skill in the art to which the present invention is directed upon review of the present specification. The claims are intended to cover such modifications and methods.

As noted above, the present invention is applicable to the identification of whether a biological sample contains antibodies against SARS-CoV-2 S glycoproteins. Accordingly, the present invention should not be considered limited of the particular examples described above, but rather should be understood to cover all aspects of the invention as set out in the attached claims.

### Enumerated Embodiments of the Disclosure

Specific enumerated embodiments <1> to <20> provided below are for illustration purposes only, and do not otherwise limit the scope of the disclosed subject matter, as defined by the claims. These enumerated embodiments encompass all combinations, sub-combinations, and multiply referenced (e.g., multiply dependent) combinations described therein.

1. A method for determining if a biological sample contains antibodies that bind to the SARS-CoV-2 Spike (S) glycoprotein, comprising:
(i) providing a surface coated with a SARS-CoV-2 S glycoprotein;
(ii) exposing the surface to the biological sample;
(iii) exposing the surface to a secondary antibody; and
(iv) detecting the secondary antibody that is bound to the surface;
   wherein the biological sample contains antibodies that bind to the SARS-CoV-2 S glycoprotein if the secondary antibody is detected.
   2. The method of enumerated embodiment 1, wherein the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 2, 4, 38, 41, 44, 48, 51, 54, 58, 61, 63, 65, 67, 73, 75, 78, 79, 82, 83, 85, 106, 108, 89, and 110, 112-115, 132, 133, 114, 138, 141, 144, 147, 151, 153, 156, 158, 174, 175, 176, 181-184, 186, 188, 190, 195, 217-228, 233-236, 243, 255-264, 273-280, 283, 284, 287, 288, 291, 292, and 294, 296, 298, 300, 302, 304, 306, 308, 310, 312, 314, 316, 318, 320, 322, 324, 236, 328, 329, 330, 331, 332 and 333.
   3. The method of any one of enumerated embodiments 1-2, wherein the SARS-CoV-2 S glycoprotein has an inactive furin cleavage site.
   4. The method of enumerated embodiment 3, wherein the SARS-CoV-2 S glycoprotein has an inactive furin cleavage site having the amino acid sequence of QQAQ (SEQ ID NO: 7).
   5. The method of any one of enumerated embodiments 1-4, wherein amino acids 973 and 974 of the SARS-CoV-2 S glycoprotein are proline, as compared to a wild-type SARS-CoV-2 S glycoprotein having the amino acid sequence of SEQ ID NO: 2.
   6. The method of any one of enumerated embodiments 1-5, wherein the antibodies that bind to the SARS-CoV-2 S glycoprotein are IgG1.
   7. The method of any one of enumerated embodiments 1-5, wherein the antibodies that bind to the SARS-CoV-2 S glycoprotein are IgG2.
   8. The method of any one of enumerated embodiments 1-5, wherein the antibodies that bind to the SARS-CoV-2 S glycoprotein are IgG3.
   9. The method of any one of enumerated embodiments 1-5, wherein the antibodies that bind to the SARS-CoV-2 S glycoprotein are IgG4.
   10. The method of any one of enumerated embodiments 1-5, wherein the secondary antibody is selected from the group consisting of an anti-human IgG antibody, an anti-human IgG1 antibody, an anti-human IgG2 antibody, an anti-human IgG3 antibody and an anti-human IgG4 antibody.
   11. The method of any one of enumerated embodiments 1-10, wherein the SARS-CoV-2 S glycoprotein is selected from a SARS-CoV-2 virus or a variant of SARS-CoV-2 or a subvariant of a variant of SARS-CoV-2.
   12. The method of enumerated embodiment 11, wherein the variant of SARS-CoV-2 is a B.1.1.7 SARS-CoV-2 strain; a B.1.351 SARS-CoV-2 strain; a P.1 SARS-CoV-2 strain; a Cal.20C SARS-CoV-2 strain; a B.1.617.2 SARS-CoV-2 strain; a B.1.525 SARS-CoV-2 strain; a B.1.526 SARS-CoV-2 strain; a B.1.617.1 SARS-CoV-2 strain; a C.37 SARS-CoV-2 strain; a B.1.621 SARS-CoV-2 strain; or a B.1.1.529 SARS-CoV-2 strain.
   13. The method of enumerated embodiment 11, wherein the variant of SARS-CoV-2 is a subvariant of a B.1.1.7 SARS-CoV-2 strain; a B.1.351 SARS-CoV-2 strain; a P.1 SARS-CoV-2 strain; a Cal.20C SARS-CoV-2 strain; a B.1.617.2 SARS-CoV-2 strain; a B.1.525 SARS-CoV-2 strain; a B.1.526 SARS-CoV-2 strain; a B.1.617.1 SARS-CoV-2 strain; a C.37 SARS-CoV-2 strain; a B.1.621 SARS-CoV-2 strain; or a B.1.1.529 SARS-CoV-2 strain.
   14. The method of any one of enumerated embodiments 1-13, wherein the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to any one of SEQ ID NOS: 2, 4, 38, 41, 44, 48, 51, 54, 58, 61, 63, 65, 67, 73, 75, 78, 79, 82, 83, 85, 106, 108, 89, and 110, 112-115, 132, 133, 114, 138, 141, 144, 147, 151, 153, 156, 158, 174, 175, 176, 181-184, 186, 188, 190, 195, 217-228, 233-236, 243, 255-264, 273-280, 283, 284, 287, 288, 291, 292, and 294, 296, 298, 300, 302, 304, 306, 308, 310, 312, 314, 316, 318, 320, 322, 324, 236, 328, 329, 330, 331, 332 and 333.
   15. The method of any one of enumerated embodiments 1-13, wherein the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to to a SARS-CoV-2 S glycoprotein from a SARS-CoV-2 S omicron variant selected from the group consisting of: BA.1, BA.2.12.1, BA.2, BA.3, BA.4, BA.5, XBB.1.5, XBB.2.3, and XBB.1.16, EG.5.1, JN.1, BQ.1.1, BF.7.
   16. The method of any one of enumerated embodiments 1-15, wherein the secondary antibody is attached to a tag.
   17. The method of enumerated embodiment 16, wherein the tag is horseradish peroxidase.
   18. The method of any one of enumerated embodiments 1-17, wherein the biological sample is serum, plasma, blood, saliva, a nasopharyngeal swab, or mucus.
   19. The method of any one of enumerated embodiments 1-18, wherein the biological sample is from a patient that has previously had COVID-19.
   20. The method of any one of enumerated embodiments 1-18, wherein the biological sample is from a patient who has been administered an immunogenic composition against a SARS-CoV-2 virus or a variant thereof.
   21. The method of any one of enumerated embodiments 1-20, wherein the SARS-CoV-2 S glycoprotein includes the transmembrane domain.

1. A method for determining if a biological sample contains antibodies that bind to the SARS-CoV-2 Spike (S) glycoprotein, comprising:
(i) providing a surface coated with a SARS-CoV-2 S glycoprotein;
(ii) exposing the surface to the biological sample;
(iii) exposing the surface to a secondary antibody; and
(iv) detecting the secondary antibody that is bound to the surface;
wherein the biological sample contains antibodies that bind to the SARS-CoV-2 S glycoprotein if the secondary antibody is detected.

2. The method of claim 1, wherein the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 2, 4, 38, 41, 44, 48, 51, 54, 58, 61, 63, 65, 67, 73, 75, 78, 79, 82, 83, 85, 106, 108, 89, and 110, 112-115, 132, 133, 114, 138, 141, 144, 147, 151, 153, 156, 158, 174, 175, 176, 181-184, 186, 188, 190, 195, 217-228, 233-236, 243, 255-264, 273-280, 283, 284, 287, 288, 291, 292, and 294, 296, 298, 300, 302, 304, 306, 308, 310, 312, 314, 316, 318, 320, 322, 324, 236, 328, 329, 330, 331, 332 and 333.

3. The method of claim 1, wherein the SARS-CoV-2 S glycoprotein has an inactive furin cleavage site.

4. The method of claim 3, wherein the SARS-CoV-2 S glycoprotein has an inactive furin cleavage site having the amino acid sequence of QQAQ (SEQ ID NO: 7).

5. The method of claim 1, wherein amino acids 973 and 974 of the SARS-CoV-2 S glycoprotein are proline, as compared to a wild-type SARS-CoV-2 S glycoprotein having the amino acid sequence of SEQ ID NO: 2.

6. The method of claim 1, wherein the antibodies that bind to the SARS-CoV-2 S glycoprotein are IgG1 or IgG2 or IgG3 or IgG4.

7. The method of claim 6, wherein the antibodies that bind to the SARS-CoV-2 S glycoprotein are IgG3.

8. The method of claim 6, wherein the antibodies that bind to the SARS-CoV-2 S glycoprotein are IgG4.

9. The method of claim 1, wherein the secondary antibody is selected from the group consisting of an anti-human IgG antibody, an anti-human IgG1 antibody, an anti-human IgG2 antibody, an anti-human IgG3 antibody and an anti-human IgG4 antibody.

10. The method of claim 1, wherein the SARS-CoV-2 S glycoprotein is selected from a SARS-CoV-2 virus or a variant of SARS-CoV-2 or a subvariant of a variant of SARS-CoV-2.

11. The method of claim 10, wherein the variant of SARS-CoV-2 is a B.1.1.7 SARS-CoV-2 strain; a B.1.351 SARS-CoV-2 strain; a P.1 SARS-CoV-2 strain; a Cal.20C SARS-CoV-2 strain; a B.1.617.2 SARS-CoV-2 strain; a B.1.525 SARS-CoV-2 strain; a B.1.526 SARS-CoV-2 strain; a B.1.617.1 SARS-CoV-2 strain; a C.37 SARS-CoV-2 strain; a B.1.621 SARS-CoV-2 strain; or a B.1.1.529 SARS-CoV-2 strain.

12. The method of claim 10, wherein the variant of SARS-CoV-2 is a subvariant of a B.1.1.7 SARS-CoV-2 strain; a B.1.351 SARS-CoV-2 strain; a P.1 SARS-CoV-2 strain; a Cal.20C SARS-CoV-2 strain; a B.1.617.2 SARS-CoV-2 strain; a B.1.525 SARS-CoV-2 strain; a B.1.526 SARS-CoV-2 strain; a B.1.617.1 SARS-CoV-2 strain; a C.37 SARS-CoV-2 strain; a B.1.621 SARS-CoV-2 strain; or a B.1.1.529 SARS-CoV-2 strain.

13. The method of claim 1, wherein the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to any one of SEQ ID NOS: 2, 4, 38, 41, 44, 48, 51, 54, 58, 61, 63, 65, 67, 73, 75, 78, 79, 82, 83, 85, 106, 108, 89, and 110, 112-115, 132, 133, 114, 138, 141, 144, 147, 151, 153, 156, 158, 174, 175, 176, 181-184, 186, 188, 190, 195, 217-228, 233-236, 243, 255-264, 273-280, 283, 284, 287, 288, 291, 292, and 294, 296, 298, 300, 302, 304, 306, 308, 310, 312, 314, 316, 318, 320, 322, 324, 236, 328, 329, 330, 331, 332 and 333.

14. The method of claim 1, wherein the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to to a SARS-CoV-2 S glycoprotein from a SARS-CoV-2 S omicron variant selected from the group consisting of: BA.1, BA.2.12.1, BA.2, BA.3, BA.4, BA.5, XBB.1.5, XBB.2.3, and XBB.1.16, EG.5.1, JN.1, BQ.1.1, BF.7.

15. The method of claim 1, wherein the secondary antibody is attached to a tag.

16. The method of claim 15, wherein the tag is horseradish peroxidase.

17. The method of claim 1, wherein the biological sample is serum, plasma, blood, saliva, a nasopharyngeal swab, or mucus.

18. The method of claim 1, wherein the biological sample is from a patient that has previously had COVID-19.

19. The method of claim 1, wherein the biological sample is from a patient who has been administered an immunogenic composition against a SARS-CoV-2 virus or a variant thereof.

20. The method of claim 1, wherein the SARS-CoV-2 S glycoprotein includes the transmembrane domain.
